SEQUENCE LISTING (162 sequences)

SEQ ID NO: 1 - DNA, 16437 bp, synthetic construct
  misc_feature 1..16437: note = Vector sequence
  source 1..16437: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggcctcgaagctggcgcgccgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaaggtaccgatccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagggccggccatcaacaactctcctggcgcaccatcgtcggctacagcctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctgagcttctagaggatccccatcgatgggccccggccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgggcccaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggcccgaacgaaacgctctacggcgctataaggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaaggtcaggcagtgatagagttggatgcctcgtcaatagaaccgtctttcgtgcaggatcgtatgcgaggggacattgacgggctccttacttcgatccgggaacaaggacagcaagtcccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgttgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggctatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagcttgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatatgcatggcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactatacgtaaccaactagtgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagcgtaccgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttag

SEQ ID NO: 2 - DNA, 557 bp, Arabidopsis thaliana
  source 1..557: mol_type = genomic DNA
taaacttgaagtctgttaaatttgatgacacctctcggtaccaatcgaagaaatctagtgaatttccgggatttttagatatctgattctgttgcgaagtttttttatccttattagtgtctgttgagtgtagattgcttggcgagtagcccacaataaagaaataaaaatggcgaatctttagctttatttactctgtttttcatcgaaacagattcagtaatatatactctgttttttcatcttacatttgtttgcaaaaccgatattatcaacttctcagtaacacaattcgttcctgaaagagtgcaaaaaaaatgttcctctacaacaccagaagtgttaagagcatcaatcatacactgaaggccaaggattcatgatcacaagtgttaaaagtttattgtctgacttaaagtaagaaacagaacattaaaagctttattattcacagttttattcatggcaagcttaggaacagtccataactaagatgaaagcggagaagaagcacaagtgcgaaaaagaaaccaaattgaaagtgttattaaaagtat

SEQ ID NO: 3 - DNA, 573 bp, Arabidopsis thaliana
  source 1..573: mol_type = genomic DNA
ttatggagattttaagtatcttttgatgatagtttacacactttgaataagctgattctactttatttctctctgtttgtttactgcttcagtgttttaagtcttctctttgtgtttgttgctgttgtgtctttgatggatttttgtgatccgatttaataaaagaagtgtgttcaaagtaattctctagtctctttatttgaaattccggggatagcagattggtttccgcaacattgactccaagtttataatgatcttgatgatggtctaaaagttttgaataagctgactctaatttactaggttctgtttgtttatataccttcagagagtttcaagtcttctcgttgtggtcttgttctgttttcttgatgtgatttcttattcattttaacaaaagaagtttgtgcaaagtcacaatgcagaaacccactttatttagattttagataagcaaagcacacagatgcattatccttaagaaaaacaaaatacaacactagaccacaacttcataaactcacaacataaccttattcaaatttcagggatatccagattggtttcaca

SEQ ID NO: 4 - DNA, 633 bp, Arabidopsis thaliana
  source 1..633: mol_type = genomic DNA
gagatgaagaaattgctcttatggttctgaaaacttctaatatgtcgagttgttctgagttttaagattttccaaaatgtctttgtctttttttttatatctttagagttacttgaacattgtgactacttctagggttgggtttgtgtcaggtctgttatatcgtgtggtgggtctgtctaatactgattcaagtttttgttattcagctaaggaacttttcttgtttctgaacaaatcttttggttccctagagtaaaacttgacttccaaaagatagacttcctaagatcactggaatagataggagtgacaacttgacgtgaaaggtagcttggctatagtaattttagatctgacaaaacgagatcaagaagttaataattttcacaacaccaatttgttatcgcttaaaggttgattgatttgttcaatagggggaatcatattctcgcatgctcgaaaaggggagagaagatggaatgaacataactttaaatcggatgtttgattcaacaacaaaactaaactcagatagagattatgaaggattccggaatctgaattacaaaagcaaaatttaaagctgaaaactcctccatggtcttactgtcacctaaagacatttctttt

SEQ ID NO: 5 - DNA, 639 bp, Arabidopsis thaliana
  source 1..639: mol_type = genomic DNA
aactgagatggaagaagaagttgcaatagtttttttaaactagtttctttctacgtactctgttttttctctgcttggttcatggtttgagtgggcaactactgatctttcccattttcctgccggatttgtagaagaataaaaaggttgaaaatgatcatcttgagatggtgatgaaactcttaagatcctcctcttctgcttctctttttatttgccgtgtcataatgaaaattgcatattcagatgggcctgtgtgttgagtaagtggacctgtgtccagagagttgattgagatgggcttcattttaggcccacatgtttatcgatcaggttcattctcttcattatatgaaagtgtcctatatcgcaacattctcttcattaatcattatattagtttatcgccacaaagttcactattcaaagattcgatttttctctcgcaaacagaaatttatattgacttttaagaaaaaatacaaagtatatctatcacacaactcacaaaagagataggtacaaacataatgacaaatcacaataagcacaccattatattaaaagtcaaatttacctttttaataagaagatacaaaaatatataaagagacgaccaagacaatttgacttgagtga

SEQ ID NO: 6 - DNA, 685 bp, Arabidopsis thaliana
  source 1..685: mol_type = genomic DNA
tgcggtatacttggcatttggttttgctgtaccctttcgaaatctttatgtcgttgtttagtttgttgtattcgactatcttcttagactttgctgaaattctcaagagatttttttactcaaacatcagtccacttgtgattggcatttatatttcaaactattgccttagttacatcttcaatagtccacttatgaacgatctttagtaaattctcttattcactcgcttactaagaattaaacctaaagagaatacagaagaattccctagagtaaggatttggtgtagtgtaaaacagactcttccctctttgtattgttcagattctggaaacataagatatgtgaatccaatcaacacagtactcgagatatgttctgataaaaaaaatgttggtactaacaatttcagtttggatgggaaggattatccgaataaagatatacctaattctgatcaaaagagagcgaaaataagtctctggagtttagattgagaaactaaatgtcaacaaaccggaaacaaaaaaaaactgtgtcaagtaacaaaaacaaaagaaaagactcaaagagagtagcagcaacgtaagatttgattccaaagtgtctcacaagggaggaatgagtaatgctactccctgttttattcactcatcacaagtccatcaatcaatctatctct

SEQ ID NO: 7 - DNA, 411 bp, Arabidopsis thaliana
  source 1..411: mol_type = genomic DNA
gttggtttcatcttattttctgcgatttttgtacttgctggatttggaatccatttgttttagctctctcgtataagattgtctcatctttgcttgttaactctatattttgaatcatcaagatatggttttgctgttaatcattgaccttcgatatttttttgccaatccgttctctctaccaacctaagaaaaaatcactaatatctcacattagagggtgcaaaatttggaaggtctatatcattgtccaattttctgagtcatacaaattctttcatatgattcattgaacaagacactcatttacttataaagcgcatttatatgttcacatgatttgtacaaaactcatgagactgcatcaagcagaaagtatttatttatctttacatgtcaaagctttgagaa

SEQ ID NO: 8 - DNA, 457 bp, Arabidopsis thaliana
  source 1..457: mol_type = genomic DNA
taacatcaaagagcaggaagtttttaacaaggaatctggtcgagacatccatgttctggaccagacagttttttggtttagccttaaaattccaaggttgtataagaagaacacactgtttgttattctttgcggttggtaaccaatatagtaatatcagtatttcgtctcaatctctcttgttcttataataatagaatgagaaatcgaaatggaattttcatgtgaagtggattagtaacattatcacaattgataaaacatttgtaaataactagacaagctttctcggctatcagttaaaatagaagttgaattaacctcacaattcgttttgtacggaactaagagatcacaaatgaaaacggaaaacaacaacaaaaatgaaaaagcgatacttttttaaaccaccctgtcatcctcatctcaaagagcttcttgacttccatgagtgcta

SEQ ID NO: 9 - DNA, 557 bp, Arabidopsis thaliana
  source 1..557: mol_type = genomic DNA
atacttttaataacactttcaatttggtttctttttcgcacttgtgcttcttctccgctttcatcttagttatggactgttcctaagcttgccatgaataaaactgtgaataataaagcttttaatgttctgtttcttactttaagtcagacaataaacttttaacacttgtgatcatgaatccttggccttcagtgtatgattgatgctcttaacacttctggtgttgtagaggaacattttttttgcactctttcaggaacgaattgtgttactgagaagttgataatatcggttttgcaaacaaatgtaagatgaaaaaacagagtatatattactgaatctgtttcgatgaaaaacagagtaaataaagctaaagattcgccatttttatttctttattgtgggctactcgccaagcaatctacactcaacagacactaataaggataaaaaaacttcgcaacagaatcagatatctaaaaatcccggaaattcactagatttcttcgattggtaccgagaggtgtcatcaaatttaacagacttcaagttta

SEQ ID NO: 10 - DNA, 573 bp, Arabidopsis thaliana
  source 1..573: mol_type = genomic DNA
tgtgaaaccaatctggatatccctgaaatttgaataaggttatgttgtgagtttatgaagttgtggtctagtgttgtattttgtttttcttaaggataatgcatctgtgtgctttgcttatctaaaatctaaataaagtgggtttctgcattgtgactttgcacaaacttcttttgttaaaatgaataagaaatcacatcaagaaaacagaacaagaccacaacgagaagacttgaaactctctgaaggtatataaacaaacagaacctagtaaattagagtcagcttattcaaaacttttagaccatcatcaagatcattataaacttggagtcaatgttgcggaaaccaatctgctatccccggaatttcaaataaagagactagagaattactttgaacacacttcttttattaaatcggatcacaaaaatccatcaaagacacaacagcaacaaacacaaagagaagacttaaaacactgaagcagtaaacaaacagagagaaataaagtagaatcagcttattcaaagtgtgtaaactatcatcaaaagatacttaaaatctccataa

SEQ ID NO: 11 - DNA, 633 bp, Arabidopsis thaliana
  source 1..633: mol_type = genomic DNA
aaaagaaatgtctttaggtgacagtaagaccatggaggagttttcagctttaaattttgcttttgtaattcagattccggaatccttcataatctctatctgagtttagttttgttgttgaatcaaacatccgatttaaagttatgttcattccatcttctctccccttttcgagcatgcgagaatatgattccccctattgaacaaatcaatcaacctttaagcgataacaaattggtgttgtgaaaattattaacttcttgatctcgttttgtcagatctaaaattactatagccaagctacctttcacgtcaagttgtcactcctatctattccagtgatcttaggaagtctatcttttggaagtcaagttttactctagggaaccaaaagatttgttcagaaacaagaaaagttccttagctgaataacaaaaacttgaatcagtattagacagacccaccacacgatataacagacctgacacaaacccaaccctagaagtagtcacaatgttcaagtaactctaaagatataaaaaaaaagacaaagacattttggaaaatcttaaaactcagaacaactcgacatattagaagttttcagaaccataagagcaatttcttcatctc

SEQ ID NO: 12 - DNA, 639 bp, Arabidopsis thaliana
  source 1..639: mol_type = genomic DNA
tcactcaagtcaaattgtcttggtcgtctctttatatatttttgtatcttcttattaaaaaggtaaatttgacttttaatataatggtgtgcttattgtgatttgtcattatgtttgtacctatctcttttgtgagttgtgtgatagatatactttgtattttttcttaaaagtcaatataaatttctgtttgcgagagaaaaatcgaatctttgaatagtgaactttgtggcgataaactaatataatgattaatgaagagaatgttgcgatataggacactttcatataatgaagagaatgaacctgatcgataaacatgtgggcctaaaatgaagcccatctcaatcaactctctggacacaggtccacttactcaacacacaggcccatctgaatatgcaattttcattatgacacggcaaataaaaagagaagcagaagaggaggatcttaagagtttcatcaccatctcaagatgatcattttcaacctttttattcttctacaaatccggcaggaaaatgggaaagatcagtagttgcccactcaaaccatgaaccaagcagagaaaaaacagagtacgtagaaagaaactagtttaaaaaaactattgcaacttcttcttccatctcagtt

SEQ ID NO: 13 - DNA, 685 bp, Arabidopsis thaliana
  source 1..685: mol_type = genomic DNA
agagatagattgattgatggacttgtgatgagtgaataaaacagggagtagcattactcattcctcccttgtgagacactttggaatcaaatcttacgttgctgctactctctttgagtcttttcttttgtttttgttacttgacacagtttttttttgtttccggtttgttgacatttagtttctcaatctaaactccagagacttattttcgctctcttttgatcagaattaggtatatctttattcggataatccttcccatccaaactgaaattgttagtaccaacattttttttatcagaacatatctcgagtactgtgttgattggattcacatatcttatgtttccagaatctgaacaatacaaagagggaagagtctgttttacactacaccaaatccttactctagggaattcttctgtattctctttaggtttaattcttagtaagcgagtgaataagagaatttactaaagatcgttcataagtggactattgaagatgtaactaaggcaatagtttgaaatataaatgccaatcacaagtggactgatgtttgagtaaaaaaatctcttgagaatttcagcaaagtctaagaagatagtcgaatacaacaaactaaacaacgacataaagatttcgaaagggtacagcaaaaccaaatgccaagtataccgca

SEQ ID NO: 14 - DNA, 411 bp, Arabidopsis thaliana
  source 1..411: mol_type = genomic DNA
ttctcaaagctttgacatgtaaagataaataaatactttctgcttgatgcagtctcatgagttttgtacaaatcatgtgaacatataaatgcgctttataagtaaatgagtgtcttgttcaatgaatcatatgaaagaatttgtatgactcagaaaattggacaatgatatagaccttccaaattttgcaccctctaatgtgagatattagtgattttttcttaggttggtagagagaacggattggcaaaaaaatatcgaaggtcaatgattaacagcaaaaccatatcttgatgattcaaaatatagagttaacaagcaaagatgagacaatcttatacgagagagctaaaacaaatggattccaaatccagcaagtacaaaaatcgcagaaaataagatgaaaccaac

SEQ ID NO: 15 - DNA, 457 bp, Arabidopsis thaliana
  source 1..457: mol_type = genomic DNA
tagcactcatggaagtcaagaagctctttgagatgaggatgacagggtggtttaaaaaagtatcgctttttcatttttgttgttgttttccgttttcatttgtgatctcttagttccgtacaaaacgaattgtgaggttaattcaacttctattttaactgatagccgagaaagcttgtctagttatttacaaatgttttatcaattgtgataatgttactaatccacttcacatgaaaattccatttcgatttctcattctattattataagaacaagagagattgagacgaaatactgatattactatattggttaccaaccgcaaagaataacaaacagtgtgttcttcttatacaaccttggaattttaaggctaaaccaaaaaactgtctggtccagaacatggatgtctcgaccagattccttgttaaaaacttcctgctctttgatgtta

SEQ ID NO: 16 - DNA, 782 bp, Oryza Sativa
  source 1..782: mol_type = genomic DNA
gcattgttttgatgctgctacctataaaagtatgagcttcagatgagaatttagagagctcccctgaggcaatgggttatgactttgttatcatcttagtactactcctatctaagggacggtgtaaggctgtaatctatgtgcggtgcaatgataaggctatcctttctgcgtttgatcatgacatctcatcgctgttcgattctttctcgctggcgtctgttatgttgaatgatgcaattcttctgtaagattccgcctttaccgaaatatctctagtatcaaatttaaattggagtacggatcctctttgaacatacggatctatttgagataattttgtctgatcatcaacgagaactcggaagcgtgccaaccttgtgggtcaattgtaacgataaagcagggagaatatagttccccctttttcctctgatgatgaaatggcaggagaagtgtagttgagcctgctgttttggtgaagactgaaatggtatcagaaagtcagaatccactataaccaatacaacagcagctgatatataatcattgatcaaggaaaacaactgcagctcatagattaatgcttactagctttgtccatcaatacatacaaaaaccagtccaacagtcatggtaactgctccaaatcaaagcacgccagaagttctcttaacgcctcacacagcaggaaacacaacaacaaactggattttatttatctacacatccatcatatttacataccaagggagaatccgcaggactcattttcctttctt

SEQ ID NO: 17 - DNA, 825 bp, Oryza Sativa
  source 1..825: mol_type = genomic DNA
aggcagcagcagtatccaactagtcatgtatccagcctaaattgaaaagtcagatcatgtgtaacatggccctcaatatttcgatgttgccttgtggaaaaaatagctggtattggtactgtgttttttactccttccagctaccgacgttcagtgttagtgcacaaaatgttcatccatggtagtttgtctacattgcagtcatgtaaagttaaacattcacgatttctgtacttttgcatgtgtaactaaatgaaatggtgtgttacattcattccttcaatcaaataatccatagaaacaaactggtttgtaactattgtaactagtcgctagtgtccatggtattttcgattgtggaacataattagtttaatctgcttcagtataacttaagccggctttatcctgtgtatgcatcagcttcgctccattgaacatccaaacatcagtataagcattttgtggcgactaatcatccggagatagggcaatgctttctaccaactgaagagtagagacgtgatgctgtgagatggaatgcaaatttcgcatattagtaagaagttggggaaggattcgaccactcactgagaagctagagcatcatggcacagtaattacaatgtaacgtagcatattcttgaacaagaacatctggtcaaataccaaccgggaaaaaatccacatcgataaagaggtgccgagtgcttacatcaacgtctttgccataatcagaacactactccgtacaagctaacacgagagcagagattacatttgcacggatgcaaaagtttccaagatagggaactggagtgaagc

SEQ ID NO: 18 - DNA, 791 bp, Oryza Sativa
  source 1..791: mol_type = genomic DNA
actgagcttttaaaagagtgaggtctaggttctgttgtctgtctgtccatcatccatgtgttgactgttgagggaactcgtttcttcttttcttcacgagatgagtttttgtgtgcctgtaatactagtttgtagcaaaggctgcgttacataaggtgatgagaattgaggtaaaatgagatctgtacactaaattcattcagactgttttggcataaagaataatttggccttctgcgatttcagaagctataaattgccatctcactaaattctccttggtcctcatggcaatgcaacgacagtgtgaagcactgaagcccgtaatgctctatcaccaccatgtacaacagaaccatatatgtccatatgtacaactcgagtgttgtttgagtggccagcaaactggctgaccaagccacacgagagagaatactataaactcaatcatacataacaagcccaagcaacagaagacagaacacaacaacactcgagtctgggaacagcaacgccgatacactacagcatactgcaacgacacaaaggaattggtaaggaaatggcaccaaaatcttgtagatctttagcgagtcattcatttatagcatatgttggaacagacatgagtcacaagatttatgatagcttagataacagatggtcggaattaaccgccgagagcctatagatgaacaaaaactctgttccaccagtgttctcatgctaaaaactttctgaaataccctaccctgttcaccgaatttctccacctgttggatagcagcatt

SEQ ID NO: 19 - DNA, 881 bp, Oryza Sativa
  source 1..881: mol_type = genomic DNA
ggtgacggcgacgcgatcgaacaggtggtgatcgatgctgcaacgtgtgtaaatatacagcgccggctgggtcaagagatggctcgggtgacgcgcgcgcggcgtgtcctggcgttggcgccggggcattctttagtttttcatcttttcatcatctcagatggtagatacaaaacagtgtatgtatgtagctctgtttctctctatagaaccccaacaaattttgttgttgatgttgtttatcttcatatgctttgatcttgaaatcgtctaccttactactgccgatcgttgctcaaaagtgtggaagtttgaagcatcttccacgggcgttgctctactcccatctcgtttcaccacgaaatccccttcgcatagaggcatctcagccgtacatctccaaacccacgttctggattcagcaaacggacggcatatgccagcggactgactaggatttgtggttcagaataaaaatatggtttgcagtgtcaattttccaggagagtgactatgtcatactaacttcatactccaataatgaagctaagctatctccatgtacatatcaaatacgaaatcatatccaagggaactaacacagtcacaaacaacaggtacacagacaattacagcacaagcgcaggagggaaataattttaactgaactaggaagaaaggaaacacaactcattttttattgatatatgttggatgaatccaataaaaccgatacaagtcacgaaaaatcagactagatgaatccttcgagatcacatgagcaaaagctttcgacgaaagctgtcctatagtcgtggaagcaataacacttgataaagataggaattcagacacgagaggttgcagactataagatgtca

SEQ ID NO: 20 - DNA, 772 bp, Oryza Sativa
  source 1..772: mol_type = genomic DNA
ccgtggcaatggctgtcatgctttggcacatactaatagcaaggtagaatggtacagctatttcattatttttgcccttgtatatttgtatcactacatgagtaaacgacgtttagttatcgatagttttgttatgagtgatgaatgatctgcatcgtactgccaatgccttgcattctcaaatggttgcacacttgcactcatacaaagttagtacactccatcatattataaattacttttttttcaagttaaatttcctgaaagtttgataaaatttatagaaaaaatataacgacgcttataacactaaattaatttcattacatctaacattaaacatattttgatttttttttgttttgtgttaaatatattactatgtttttctataaacttgattaaacatataaaagtttaacttcaaaaaaaagttaaaatgacttgtaatataaaacgtaggcagtacaatgcgaatgtagggtactccatccagctgaggtaaaccaactccaatatatatacaaacacaaacaacgtacccaatttttactgttaaaatacaggcacaatgcctggtatcacacgttattaagtagacagactcgataaccatgacacggacagggacttcttgccactggtttacgcacggttaatattacagaccacacatagagagacggcttagctatttgcaaataagcttgacaagatagatgatgctccaaaaggatgcgatctcagcagttgagtacttacgctggttcat

SEQ ID NO: 21 - DNA, 827 bp, Oryza Sativa
  source 1..827: mol_type = genomic DNA
acaccacctgctgtctgcgcttgtttgttgccacggctgcgctacaaacacaagccgtatcatctgccgaacagctccatcgttagcttcttttgatgccttacatctatcgctggacgtttatcgtgcgtacttgtcccatacagggattgtgtttggatgatttgtagagagggtactgtatggcataagatccttcatattaatgttctgtgggtcacatatcaacttgctattattgtattcagcggttctctctctattttgccttaagctgtggtaccattttaagtccagtgcttgcttttgattgcttttctgagctttccaggacatggaatcgagcctgtgaatgtcgtcggccggtttatcctttcgactaataatttaaattgtccagtgttggttaggaatggagtatcagattctggcggtttgcatgctgtttggtcggcgacgtgccttatcctgaagatttatatatagtactttgattgatgtcaagaaggaaatttctttaagaaactttttacaaatagcgattggtagaactgattaacaacatccacggctcagaaaaggaacgcaaacatatttgattcttccctaaattaatttgccattgttatttttacttgaccatccgtcaaaatttacatgaatacacaacgcacggacgaacgatttattcatgtgaaggcatgatttacatccttaaaattctctcgcatctagatatagctagagagtggccagctgctgactatgcatggagctggcacggcagtagtgtacaatgctactgtaattttgtattgctacaagta

SEQ ID NO: 22 - DNA, 770 bp, Oryza Sativa
  source 1..770: mol_type = genomic DNA
gctgaaatttggttatctgttatgagcatatctgatttcgatacccactgttatgaaactgaataaaccgcattcctgtatgctgggaattttctcgttagtgtacgctccaatactcgtgccgttttcaaagacaagcagtgatatgcaaagcaatgctgtgtatcttgtgtgttacatcaggagttttttttaatggtgatttcgttcatagttttcagacatctgtttccgtcacacgttcgtgggaatgacttttttcatatgaaatgcgcccatggctgcgcctgaaataattttacttgtcccgtttgggatgaatgaattcttttcctgttcgttgttcagtaattcacacagagtcataaacataccggtggaaacatgctcacaaagaaagaaacatcatgaagtcaggctaaggataagaatagccagcacacctagagcaaaattttactaacatcagatgcaagaaagtctacatgagttctaaaaaggaaactggggaaaactaatttacaccaattatcactaatagtacactaaatataaacgaccaagaagggctctaggaacccattttgtatctagcacccgaacgttgaacctttctcccgataaaagagggatcaggcgtgcaacagactggctctcacgccagggaaaccacaggtacaaaagaggcagcgaaagaagaaacaaaacttttcctccggggcgaacatatacaacttgagaggaaaaatacacaatggccgagagacaaa

SEQ ID NO: 23 - DNA, 814 bp, Oryza Sativa
  source 1..814: mol_type = genomic DNA
agtgttacttattgttgctactatccatattcgctaccatgcttatcatcgctagattaaaggacatgtattccagtatggtcgtgctttttgcttggtactctggtcacatttgtgatgaatgttcgcagttttcgtggtcatggtctggattctacattccacctaaagaagtttttacctggttatttaatccttgagttgttgaattagctcatggtaattgtcagtagtcttccacttggttctctctgtggatgatcgttcttgttctctgtttgcgctgtggctgattgttctcatgtttttaaggtttttgggttagtcactggtcgcttcgtcactgtgagccaaaccatatgccggtagttttcttcttcaccatcatccgtttgatcctgactcgcggatctcctaatccaacttctctcttttttttttttaatccaaactcacccaacgttggaaacatagcttggaaccccttgaaattgaacagaatttatagcaggcaaaaatgtaagaaaccacgctagtgtagcaggcttacagcggggcaaagcagcttttgttaagcaaccacttacgttagagattcagagataagcgatgttactttgtttttaccaacatttacatacaagcggtacaacacatattgccagaaaccacttggttttccagccataatttttgacaattcatgacgcgcgcgcgcacacacaatgctggattcacagggtcacacagccatgagccatcagactggaagggctctgtttgttcatcacatagtcacagatcgccttggcct

SEQ ID NO: 24 - DNA, 834 bp, Oryza Sativa
  source 1..834: mol_type = genomic DNA
ttggcagcctacccactctggtaggaaaaatggaaggggcaagaagatacagtcgatgatatttgatgtgctacataccaattgtaatatttcattcatatctagagctagaaggatattcacttgtatttcagactctggttcacggtttaaattgatattgtggtagaatgctttcttggatgggtatatagtgtgactcgttaaaaatatgacaaaacggttcagctactctttacaagtggtacggcaaattgtagacccaattgcacttggcggtgagctgctttgttggagcaacagggcactgtctgcttgcgtggatgaaaagctgagtcatctgaagctagtgaacattttttcgctggctcgctgcgtgtgttttttctctgattgtggcccggttgattatgaaaatgaccagtaagtaaagtcatccgaccctgctgatttgtttgttttgcctaacctagctcatgctcggggtcgtgaatcgtgctacacttgttgcagctttcgttttcttgaagagttggaaggtgtcagcttagcttcctgtgcgtactagtattgcagaagaaaacgtaaaactgtaccagagcaagaagactttgacataaatttgtaacaaggcagataggtggtacaaatcaaggcactcagctgaacagctcaactgaaatgcagaactgaattgaagagtaaaaatgatccatgcatgacagacaaaccagtggacactgactgaagatggagcaatgaacaaaataaaatcaagacttgtttttattggcgcatgcatatttagggtgatatgatgcatgacgaaatgaa

SEQ ID NO: 25 - DNA, 740 bp, Oryza Sativa
  source 1..740: mol_type = genomic DNA
ggtgttccatatagatgatcagatttctataaccacatgagtacaatgtagagaattcaaatgttttgtaaccacgtgagctttgtacgctatttgcaccacagttctccctgtatgttgttggctcttttacaattaagaataatgcatgttgaagtatgagtcttcctgtttgcttctccttgatgatgaaacgctgctgcatcaactgattatttttcagagaataaacgataaacaaagcacggtgaagtatcgtgtaacccaccataagtggtagagccgtagaggtaggactgtgcatgctgaattgtaattaccatggttagttcagcagaatttgcaaaaagaggacaacagaattagaccatcgtcatgaattaatcaaggtacaaaccaatggttgctggtacatcagtagcaaatggctgtagcactagcgtgcccatatttatattccaagcctccaaggtatacaatacatatttgatgtaccgaacaactgaaaaaggcgcttggtttggaagcgcactatttttaaatttataggataacttctgacagcctttcttctaaatgaacttttggcaaccgctctgaagccgtgtaacacaatccaacaaagcaatttgcagtcaaaatttcgggcatgtgccgttctagttagaatttaggatgtgactcactaagtaattgtgattgtttctctatgcaaacaccagccaacagtattgatttca

SEQ ID NO: 26 - DNA, 782 bp, Oryza Sativa
  source 1..782: mol_type = genomic DNA
aagaaaggaaaatgagtcctgcggattctcccttggtatgtaaatatgatggatgtgtagataaataaaatccagtttgttgttgtgtttcctgctgtgtgaggcgttaagagaacttctggcgtgctttgatttggagcagttaccatgactgttggactggtttttgtatgtattgatggacaaagctagtaagcattaatctatgagctgcagttgttttccttgatcaatgattatatatcagctgctgttgtattggttatagtggattctgactttctgataccatttcagtcttcaccaaaacagcaggctcaactacacttctcctgccatttcatcatcagaggaaaaagggggaactatattctccctgctttatcgttacaattgacccacaaggttggcacgcttccgagttctcgttgatgatcagacaaaattatctcaaatagatccgtatgttcaaagaggatccgtactccaatttaaatttgatactagagatatttcggtaaaggcggaatcttacagaagaattgcatcattcaacataacagacgccagcgagaaagaatcgaacagcgatgagatgtcatgatcaaacgcagaaaggatagccttatcattgcaccgcacatagattacagccttacaccgtcccttagataggagtagtactaagatgataacaaagtcataacccattgcctcaggggagctctctaaattctcatctgaagctcatacttttataggtagcagcatcaaaacaatgc

SEQ ID NO: 27 - DNA, 825 bp, Oryza Sativa
  source 1..825: mol_type = genomic DNA
gcttcactccagttccctatcttggaaacttttgcatccgtgcaaatgtaatctctgctctcgtgttagcttgtacggagtagtgttctgattatggcaaagacgttgatgtaagcactcggcacctctttatcgatgtggattttttcccggttggtatttgaccagatgttcttgttcaagaatatgctacgttacattgtaattactgtgccatgatgctctagcttctcagtgagtggtcgaatccttccccaacttcttactaatatgcgaaatttgcattccatctcacagcatcacgtctctactcttcagttggtagaaagcattgccctatctccggatgattagtcgccacaaaatgcttatactgatgtttggatgttcaatggagcgaagctgatgcatacacaggataaagccggcttaagttatactgaagcagattaaactaattatgttccacaatcgaaaataccatggacactagcgactagttacaatagttacaaaccagtttgtttctatggattatttgattgaaggaatgaatgtaacacaccatttcatttagttacacatgcaaaagtacagaaatcgtgaatgtttaactttacatgactgcaatgtagacaaactaccatggatgaacattttgtgcactaacactgaacgtcggtagctggaaggagtaaaaaacacagtaccaataccagctattttttccacaaggcaacatcgaaatattgagggccatgttacacatgatctgacttttcaatttaggctggatacatgactagttggatactgctgctgcct

SEQ ID NO: 28 - DNA, 776 bp, Oryza Sativa
  source 1..776: mol_type = genomic DNA
aatgctgctatccaacaggtggagaaattcggtgaacagggtagggtatttcagaaagtttttagcatgagaacactggtggaacagagtttttgttcatctataggctctcggcggttaattccgaccatctgttatctaagctatcataaatcttgtgactcatgtctgttccaacatatgctataaatgaatgactcgctaaagatctacaagattttggtgccatttccttaccaattcctttgtgtcgttgcagtatgctgtagtgtatcggcgttgctgttcccagactcgagtgttgttgtgttctgtcttctgttgcttgggcttgttatgtatgattgagtttatagtattctctctcgtgtggcttggtcagccagtttgctggccactcaaacaacactcgagttgtacatatggacatatatggttctgttgtacatggtggtgatagagcattacgggcttcagtgcttcacactgtcgttgcattgccatgaggaccaaggagaatttagtgagatggcaatttatagcttctgaaatcgcagaaggccaaattattctttatgccaaaacagtctgaatgaatttagtgtacagatctcattttacctcaattctcatcaccttatgtaacgcagcctttgctacaaactagtattacaggcacacaaaaactcatctcgtgaagaaaagaagaaacgagttccctcaacagtcaacacatggatgatggacagacagacaacagaacctagacctcactc

SEQ ID NO: 29 - DNA, 881 bp, Oryza Sativa
  source 1..881: mol_type = genomic DNA
tgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcacc

SEQ ID NO: 30 - DNA, 772 bp, Oryza Sativa
  source 1..772: mol_type = genomic DNA
atgaaccagcgtaagtactcaactgctgagatcgcatccttttggagcatcatctatcttgtcaagcttatttgcaaatagctaagccgtctctctatgtgtggtctgtaatattaaccgtgcgtaaaccagtggcaagaagtccctgtccgtgtcatggttatcgagtctgtctacttaataacgtgtgataccaggcattgtgcctgtattttaacagtaaaaattgggtacgttgtttgtgtttgtatatatattggagttggtttacctcagctggatggagtaccctacattcgcattgtactgcctacgttttatattacaagtcattttaacttttttttgaagttaaacttttatatgtttaatcaagtttatagaaaaacatagtaatatatttaacacaaaacaaaaaaaaatcaaaatatgtttaatgttagatgtaatgaaattaatttagtgttataagcgtcgttatattttttctataaattttatcaaactttcaggaaatttaacttgaaaaaaaagtaatttataatatgatggagtgtactaactttgtatgagtgcaagtgtgcaaccatttgagaatgcaaggcattggcagtacgatgcagatcattcatcactcataacaaaactatcgataactaaacgtcgtttactcatgtagtgatacaaatatacaagggcaaaaataatgaaatagctgtaccattctaccttgctattagtatgtgccaaagcatgacagccattgccacgg

SEQ ID NO: 31 - DNA, 827 bp, Oryza Sativa
  source 1..827: mol_type = genomic DNA
tacttgtagcaatacaaaattacagtagcattgtacactactgccgtgccagctccatgcatagtcagcagctggccactctctagctatatctagatgcgagagaattttaaggatgtaaatcatgccttcacatgaataaatcgttcgtccgtgcgttgtgtattcatgtaaattttgacggatggtcaagtaaaaataacaatggcaaattaatttagggaagaatcaaatatgtttgcgttccttttctgagccgtggatgttgttaatcagttctaccaatcgctatttgtaaaaagtttcttaaagaaatttccttcttgacatcaatcaaagtactatatataaatcttcaggataaggcacgtcgccgaccaaacagcatgcaaaccgccagaatctgatactccattcctaaccaacactggacaatttaaattattagtcgaaaggataaaccggccgacgacattcacaggctcgattccatgtcctggaaagctcagaaaagcaatcaaaagcaagcactggacttaaaatggtaccacagcttaaggcaaaatagagagagaaccgctgaatacaataatagcaagttgatatgtgacccacagaacattaatatgaaggatcttatgccatacagtaccctctctacaaatcatccaaacacaatccctgtatgggacaagtacgcacgataaacgtccagcgatagatgtaaggcatcaaaagaagctaacgatggagctgttcggcagatgatacggcttgtgtttgtagcgcagccgtggcaacaaacaagcgcagacagcaggtggtgt

SEQ ID NO: 32 - DNA, 770 bp, Oryza Sativa
  source 1..770: mol_type = genomic DNA
tttgtctctcggccattgtgtatttttcctctcaagttgtatatgttcgccccggaggaaaagttttgtttcttctttcgctgcctcttttgtacctgtggtttccctggcgtgagagccagtctgttgcacgcctgatccctcttttatcgggagaaaggttcaacgttcgggtgctagatacaaaatgggttcctagagcccttcttggtcgtttatatttagtgtactattagtgataattggtgtaaattagttttccccagtttcctttttagaactcatgtagactttcttgcatctgatgttagtaaaattttgctctaggtgtgctggctattcttatccttagcctgacttcatgatgtttctttctttgtgagcatgtttccaccggtatgtttatgactctgtgtgaattactgaacaacgaacaggaaaagaattcattcatcccaaacgggacaagtaaaattatttcaggcgcagccatgggcgcatttcatatgaaaaaagtcattcccacgaacgtgtgacggaaacagatgtctgaaaactatgaacgaaatcaccattaaaaaaaactcctgatgtaacacacaagatacacagcattgctttgcatatcactgcttgtctttgaaaacggcacgagtattggagcgtacactaacgagaaaattcccagcatacaggaatgcggtttattcagtttcataacagtgggtatcgaaatcagatatgctcataacagataaccaaatttcagc

SEQ ID NO: 33 - DNA, 814 bp, Oryza Sativa
  source 1..814: mol_type = genomic DNA
aggccaaggcgatctgtgactatgtgatgaacaaacagagcccttccagtctgatggctcatggctgtgtgaccctgtgaatccagcattgtgtgtgcgcgcgcgcgtcatgaattgtcaaaaattatggctggaaaaccaagtggtttctggcaatatgtgttgtaccgcttgtatgtaaatgttggtaaaaacaaagtaacatcgcttatctctgaatctctaacgtaagtggttgcttaacaaaagctgctttgccccgctgtaagcctgctacactagcgtggtttcttacatttttgcctgctataaattctgttcaatttcaaggggttccaagctatgtttccaacgttgggtgagtttggattaaaaaaaaaaaagagagaagttggattaggagatccgcgagtcaggatcaaacggatgatggtgaagaagaaaactaccggcatatggtttggctcacagtgacgaagcgaccagtgactaacccaaaaaccttaaaaacatgagaacaatcagccacagcgcaaacagagaacaagaacgatcatccacagagagaaccaagtggaagactactgacaattaccatgagctaattcaacaactcaaggattaaataaccaggtaaaaacttctttaggtggaatgtagaatccagaccatgaccacgaaaactgcgaacattcatcacaaatgtgaccagagtaccaagcaaaaagcacgaccatactggaatacatgtcctttaatctagcgatgataagcatggtagcgaatatggatagtagcaacaataagtaacact

SEQ ID NO: 34 - DNA, 834 bp, Oryza Sativa
  source 1..834: mol_type = genomic DNA
ttcatttcgtcatgcatcatatcaccctaaatatgcatgcgccaataaaaacaagtcttgattttattttgttcattgctccatcttcagtcagtgtccactggtttgtctgtcatgcatggatcatttttactcttcaattcagttctgcatttcagttgagctgttcagctgagtgccttgatttgtaccacctatctgccttgttacaaatttatgtcaaagtcttcttgctctggtacagttttacgttttcttctgcaatactagtacgcacaggaagctaagctgacaccttccaactcttcaagaaaacgaaagctgcaacaagtgtagcacgattcacgaccccgagcatgagctaggttaggcaaaacaaacaaatcagcagggtcggatgactttacttactggtcattttcataatcaaccgggccacaatcagagaaaaaacacacgcagcgagccagcgaaaaaatgttcactagcttcagatgactcagcttttcatccacgcaagcagacagtgccctgttgctccaacaaagcagctcaccgccaagtgcaattgggtctacaatttgccgtaccacttgtaaagagtagctgaaccgttttgtcatatttttaacgagtcacactatatacccatccaagaaagcattctaccacaatatcaatttaaaccgtgaaccagagtctgaaatacaagtgaatatccttctagctctagatatgaatgaaatattacaattggtatgtagcacatcaaatatcatcgactgtatcttcttgccccttccatttttcctaccagagtgggtaggctgccaa

SEQ ID NO: 35 - DNA, 740 bp, Oryza Sativa
  source 1..740: mol_type = genomic DNA
tgaaatcaatactgttggctggtgtttgcatagagaaacaatcacaattacttagtgagtcacatcctaaattctaactagaacggcacatgcccgaaattttgactgcaaattgctttgttggattgtgttacacggcttcagagcggttgccaaaagttcatttagaagaaaggctgtcagaagttatcctataaatttaaaaatagtgcgcttccaaaccaagcgcctttttcagttgttcggtacatcaaatatgtattgtataccttggaggcttggaatataaatatgggcacgctagtgctacagccatttgctactgatgtaccagcaaccattggtttgtaccttgattaattcatgacgatggtctaattctgttgtcctctttttgcaaattctgctgaactaaccatggtaattacaattcagcatgcacagtcctacctctacggctctaccacttatggtgggttacacgatacttcaccgtgctttgtttatcgtttattctctgaaaaataatcagttgatgcagcagcgtttcatcatcaaggagaagcaaacaggaagactcatacttcaacatgcattattcttaattgtaaaagagccaacaacatacagggagaactgtggtgcaaatagcgtacaaagctcacgtggttacaaaacatttgaattctctacattgtactcatgtggttatagaaatctgatcatctatatggaacacc

SEQ ID NO: 36 - DNA, 330 bp, Solanum tuberosum
  source 1..330: mol_type = genomic DNA
cctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 37 - DNA, 40 bp, synthetic construct
  misc_feature 1..40: note = T1 fwd primer
  source 1..40: mol_type = other DNA
aggcaaacaaggtacctaaacttgaagtctgttaaatttg

SEQ ID NO: 38 - DNA, 42 bp, synthetic construct
  misc_feature 1..42: note = T1 rev primer
  source 1..42: mol_type = other DNA
gccatggatcggtaccatacttttaataacactttcaatttg

SEQ ID NO: 39 - DNA, 41 bp, synthetic construct
  misc_feature 1..41: note = T2 fwd primer
  source 1..41: mol_type = other DNA
aggcaaacaaggtaccttatggagattttaagtatcttttg

SEQ ID NO: 40 - DNA, 37 bp, synthetic construct
  misc_feature 1..37: note = T2 rev primer
  source 1..37: mol_type = other DNA
gccatggatcggtacctgtgaaaccaatctggatatc

SEQ ID NO: 41 - DNA, 39 bp, synthetic construct
  misc_feature 1..39: note = T3 fwd primer
  source 1..39: mol_type = other DNA
aggcaaacaaggtaccgagatgaagaaattgctcttatg

SEQ ID NO: 42 - DNA, 38 bp, synthetic construct
  misc_feature 1..38: note = T3 rev primer
  source 1..38: mol_type = other DNA
gccatggatcggtaccaaaagaaatgtctttaggtgac

SEQ ID NO: 43 - DNA, 39 bp, synthetic construct
  misc_feature 1..39: note = T4 fwd primer
  source 1..39: mol_type = other DNA
aggcaaacaaggtaccaactgagatggaagaagaagttg

SEQ ID NO: 44 - DNA, 38 bp, synthetic construct
  misc_feature 1..38: note = T4 rev primer
  source 1..38: mol_type = other DNA
gccatggatcggtacctcactcaagtcaaattgtcttg

SEQ ID NO: 45 - DNA, 36 bp, synthetic construct
  misc_feature 1..36: note = T5 fwd primer
  source 1..36: mol_type = other DNA
aggcaaacaaggtacctgcggtatacttggcatttg

SEQ ID NO: 46 - DNA, 38 bp, synthetic construct
  misc_feature 1..38: note = T5 rev primer
  source 1..38: mol_type = other DNA
gccatggatcggtaccagagatagattgattgatggac

SEQ ID NO: 47 - DNA, 39 bp, synthetic construct
  misc_feature 1..39: note = T6 fwd primer
  source 1..39: mol_type = other DNA
aggcaaacaaggtaccgttggtttcatcttattttctgc

SEQ ID NO: 48 - DNA, 40 bp, synthetic construct
  misc_feature 1..40: note = T6 rev primer
  source 1..40: mol_type = other DNA
gccatggatcggtaccttctcaaagctttgacatgtaaag

SEQ ID NO: 49 - DNA, 37 bp, synthetic construct
  misc_feature 1..37: note = T7 fwd primer
  source 1..37: mol_type = other DNA
aggcaaacaaggtacctaacatcaaagagcaggaagt

SEQ ID NO: 50 - DNA, 36 bp, synthetic construct
  misc_feature 1..36: note = T7 rev primer
  source 1..36: mol_type = other DNA
gccatggatcggtacctagcactcatggaagtcaag

SEQ ID NO: 51 - DNA, 42 bp, synthetic construct
  misc_feature 1..42: note = T8 fwd primer
  source 1..42: mol_type = other DNA
aggcaaacaaggtaccatacttttaataacactttcaatttg

SEQ ID NO: 52 - DNA, 40 bp, synthetic construct
  misc_feature 1..40: note = T8 rev primer
  source 1..40: mol_type = other DNA
gccatggatcggtacctaaacttgaagtctgttaaatttg

SEQ ID NO: 53 - DNA, 39 bp, synthetic construct
  misc_feature 1..39: note = T9 fwd primer
  source 1..39: mol_type = other DNA
aggcaaacaaggtacctgtgaaaccaatctggatatccc

SEQ ID NO: 54 - DNA, 38 bp, synthetic construct
  misc_feature 1..38: note = T9 rev primer
  source 1..38: mol_type = other DNA
gccatggatcggtaccttatggagattttaagtatctt

SEQ ID NO: 55 - DNA, 40 bp, synthetic construct
  misc_feature 1..40: note = T10 fwd primer
  source 1..40: mol_type = other DNA
aggcaaacaaggtaccaaaagaaatgtctttaggtgacag

SEQ ID NO: 56 - DNA, 39 bp, synthetic construct
  misc_feature 1..39: note = T10 rev primer
  source 1..39: mol_type = other DNA
gccatggatcggtaccgagatgaagaaattgctcttatg

SEQ ID NO: 57 - DNA, 39 bp, synthetic construct
  misc_feature 1..39: note = T11 fwd primer
  source 1..39: mol_type = other DNA
aggcaaacaaggtacctcactcaagtcaaattgtcttgg

SEQ ID NO: 58 - DNA, 40 bp, synthetic construct
  misc_feature 1..40: note = T11 rev primer
  source 1..40: mol_type = other DNA
gccatggatcggtaccaactgagatggaagaagaagttgc

SEQ ID NO: 59 - DNA, 39 bp, synthetic construct
  misc_feature 1..39: note = T12 fwd primer
  source 1..39: mol_type = other DNA
aggcaaacaaggtaccagagatagattgattgatggact

SEQ ID NO: 60 - DNA, 38 bp, synthetic construct
  misc_feature 1..38: note = T12 rev primer
  source 1..38: mol_type = other DNA
gccatggatcggtacctgcggtatacttggcatttggt

SEQ ID NO: 61 - DNA, 37 bp, synthetic construct
  misc_feature 1..37: note = T13 fwd primer
  source 1..37: mol_type = other DNA
aggcaaacaaggtaccttctcaaagctttgacatgta

SEQ ID NO: 62 - DNA, 39 bp, synthetic construct
  misc_feature 1..39: note = T13 rev primer
  source 1..39: mol_type = other DNA
gccatggatcggtaccgttggtttcatcttattttctgc

SEQ ID NO: 63 - DNA, 36 bp, synthetic construct
  misc_feature 1..36: note = T14 fwd primer
  source 1..36: mol_type = other DNA
aggcaaacaaggtacctagcactcatggaagtcaag

SEQ ID NO: 64 - DNA, 36 bp, synthetic construct
  misc_feature 1..36: note = T14 rev primer
  source 1..36: mol_type = other DNA
gccatggatcggtacctaacatcaaagagcaggaag

SEQ ID NO: 65 - DNA, 38 bp, synthetic construct
  misc_feature 1..38: note = T15 fwd primer
  source 1..38: mol_type = other DNA
aggcaaacaaggtaccgcattgttttgatgctgctacc

SEQ ID NO: 66 - DNA, 38 bp, synthetic construct
  misc_feature 1..38: note = T15 rev primer
  source 1..38: mol_type = other DNA
gccatggatcggtaccaagaaaggaaaatgagtcctgc

SEQ ID NO: 67 - DNA, 38 bp, synthetic construct
  misc_feature 1..38: note = T16 fwd primer
  source 1..38: mol_type = other DNA
aggcaaacaaggtaccaggcagcagcagtatccaacta

SEQ ID NO: 68 - DNA, 38 bp, synthetic construct
  misc_feature 1..38: note = T16 rev primer
  source 1..38: mol_type = other DNA
gccatggatcggtaccgcttcactccagttccctatct

SEQ ID NO: 69 - DNA, 38 bp, synthetic construct
  misc_feature 1..38: note = T17 fwd primer
  source 1..38: mol_type = other DNA
aggcaaacaaggtaccgagtgaggtctaggttctgttg

SEQ ID NO: 70 - DNA, 38 bp, synthetic construct
  misc_feature 1..38: note = T17 rev primer
  source 1..38: mol_type = other DNA
gccatggatcggtaccaatgctgctatccaacaggtgg

SEQ ID NO: 71 - DNA, 38 bp, synthetic construct
  misc_feature 1..38: note = T18 fwd primer
  source 1..38: mol_type = other DNA
aggcaaacaaggtaccggtgacggcgacgcgatcgaac

SEQ ID NO: 72 - DNA, 40 bp, synthetic construct
  misc_feature 1..40: note = T18 rev primer
  source 1..40: mol_type = other DNA
gccatggatcggtacctgacatcttatagtctgcaacctc

SEQ ID NO: 73 - DNA, 36 bp, synthetic construct
  misc_feature 1..36: note = T19 fwd primer
  source 1..36: mol_type = other DNA
aggcaaacaaggtaccccgtggcaatggctgtcatg

SEQ ID NO: 74 - DNA, 39 bp, synthetic construct
  misc_feature 1..39: note = T19 rev primer
  source 1..39: mol_type = other DNA
gccatggatcggtaccatgaaccagcgtaagtactcaac

SEQ ID NO: 75 - DNA, 38 bp, synthetic construct
  misc_feature 1..38: note = T20 fwd primer
  source 1..38: mol_type = other DNA
aggcaaacaaggtaccacaccacctgctgtctgcgctt

SEQ ID NO: 76 - DNA, 41 bp, synthetic construct
  misc_feature 1..41: note = T20 rev primer
  source 1..41: mol_type = other DNA
gccatggatcggtacctacttgtagcaatacaaaattacag

SEQ ID NO: 77 - DNA, 38 bp, synthetic construct
  misc_feature 1..38: note = T21 fwd primer
  source 1..38: mol_type = other DNA
aggcaaacaaggtaccgctgaaatttggttatctgtta

SEQ ID NO: 78 - DNA, 38 bp, synthetic construct
  misc_feature 1..38: note = T21 rev primer
  source 1..38: mol_type = other DNA
gccatggatcggtacctttgtctctcggccattgtgta

SEQ ID NO: 79 - DNA, 39 bp, synthetic construct
  misc_feature 1..39: note = T22 fwd primer
  source 1..39: mol_type = other DNA
aggcaaacaaggtaccagtgttacttattgttgctacta

SEQ ID NO: 80 - DNA, 38 bp, synthetic construct
  misc_feature 1..38: note = T22 rev primer
  source 1..38: mol_type = other DNA
gccatggatcggtaccaggccaaggcgatctgtgacta

SEQ ID NO: 81 - DNA, 38 bp, synthetic construct
  misc_feature 1..38: note = T23 fwd primer
  source 1..38: mol_type = other DNA
aggcaaacaaggtaccttggcagcctacccactctggt

SEQ ID NO: 82 - DNA, 38 bp, synthetic construct
  misc_feature 1..38: note = T23 rev primer
  source 1..38: mol_type = other DNA
gccatggatcggtaccttcatttcgtcatgcatcatat

SEQ ID NO: 83 - DNA, 38 bp, synthetic construct
  misc_feature 1..38: note = T24 fwd primer
  source 1..38: mol_type = other DNA
aggcaaacaaggtaccggtgttccatatagatgatcag

SEQ ID NO: 84 - DNA, 38 bp, synthetic construct
  misc_feature 1..38: note = T24 rev primer
  source 1..38: mol_type = other DNA
gccatggatcggtacctgaaatcaatactgttggctgg

SEQ ID NO: 85 - DNA, 38 bp, synthetic construct
  misc_feature 1..38: note = T25 fwd primer
  source 1..38: mol_type = other DNA
aggcaaacaaggtaccaagaaaggaaaatgagtcctgc

SEQ ID NO: 86 - DNA, 38 bp, synthetic construct
  misc_feature 1..38: note = T25 rev primer
  source 1..38: mol_type = other DNA
gccatggatcggtaccgcattgttttgatgctgctacc

SEQ ID NO: 87 - DNA, 38 bp, synthetic construct
  misc_feature 1..38: note = T26 fwd primer
  source 1..38: mol_type = other DNA
aggcaaacaaggtaccgcttcactccagttccctatct

SEQ ID NO: 88 - DNA, 38 bp, synthetic construct
  misc_feature 1..38: note = T26 rev primer
  source 1..38: mol_type = other DNA
gccatggatcggtaccaggcagcagcagtatccaacta

SEQ ID NO: 89 - DNA, 38 bp, synthetic construct
  misc_feature 1..38: note = T27 fwd primer
  source 1..38: mol_type = other DNA
aggcaaacaaggtaccaatgctgctatccaacaggtgg

SEQ ID NO: 90 - DNA, 38 bp, synthetic construct
  misc_feature 1..38: note = T27 rev primer
  source 1..38: mol_type = other DNA
gccatggatcggtaccgagtgaggtctaggttctgttg

SEQ ID NO: 91 - DNA, 38 bp, synthetic construct
  misc_feature 1..38: note = T28 fwd primer
  source 1..38: mol_type = other DNA
aggcaaacaaggtacctgacatcttatagtctgcaacc

SEQ ID NO: 92 - DNA, 36 bp, synthetic construct
  misc_feature 1..36: note = T28 rev primer
  source 1..36: mol_type = other DNA
gccatggatcggtaccggtgacggcgacgcgatcga

SEQ ID NO: 93 - DNA, 38 bp, synthetic construct
  misc_feature 1..38: note = T29 fwd primer
  source 1..38: mol_type = other DNA
aggcaaacaaggtaccatgaaccagcgtaagtactcaa

SEQ ID NO: 94 - DNA, 36 bp, synthetic construct
  misc_feature 1..36: note = T29 rev primer
  source 1..36: mol_type = other DNA
gccatggatcggtaccccgtggcaatggctgtcatg

SEQ ID NO: 95 - DNA, 40 bp, synthetic construct
  misc_feature 1..40: note = T30 fwd primer
  source 1..40: mol_type = other DNA
aggcaaacaaggtacctacttgtagcaatacaaaattaca

SEQ ID NO: 96 - DNA, 38 bp, synthetic construct
  misc_feature 1..38: note = T30 rev primer
  source 1..38: mol_type = other DNA
gccatggatcggtaccacaccacctgctgtctgcgctt

SEQ ID NO: 97 - DNA, 38 bp, synthetic construct
  misc_feature 1..38: note = T31 fwd primer
  source 1..38: mol_type = other DNA
aggcaaacaaggtacctttgtctctcggccattgtgta

SEQ ID NO: 98 - DNA, 40 bp, synthetic construct
  misc_feature 1..40: note = T31 rev primer
  source 1..40: mol_type = other DNA
gccatggatcggtaccgctgaaatttggttatctgttatg

SEQ ID NO: 99 - DNA, 38 bp, synthetic construct
  misc_feature 1..38: note = T32 fwd primer
  source 1..38: mol_type = other DNA
aggcaaacaaggtaccaggccaaggcgatctgtgacta

SEQ ID NO: 100 - DNA, 40 bp, synthetic construct
  misc_feature 1..40: note = T32 rev primer
  source 1..40: mol_type = other DNA
gccatggatcggtaccagtgttacttattgttgctactat

SEQ ID NO: 101 - DNA, 38 bp, synthetic construct
  misc_feature 1..38: note = T33 fwd primer
  source 1..38: mol_type = other DNA
aggcaaacaaggtaccttcatttcgtcatgcatcatat

SEQ ID NO: 102 - DNA, 38 bp, synthetic construct
  misc_feature 1..38: note = T33 rev primer
  source 1..38: mol_type = other DNA
gccatggatcggtaccttggcagcctacccactctggt

SEQ ID NO: 103 - DNA, 38 bp, synthetic construct
  misc_feature 1..38: note = T34 fwd primer
  source 1..38: mol_type = other DNA
aggcaaacaaggtacctgaaatcaatactgttggctgg

SEQ ID NO: 104 - DNA, 38 bp, synthetic construct
  misc_feature 1..38: note = T34 rev primer
  source 1..38: mol_type = other DNA
gccatggatcggtaccggtgttccatatagatgatcag

SEQ ID NO: 105 - DNA, 36 bp, synthetic construct
  misc_feature 1..36: note = PinII fwd primer
  source 1..36: mol_type = other DNA
aggcaaacaaggtacccctcggtgacgtggggcaac

SEQ ID NO: 106 - DNA, 36 bp, synthetic construct
  misc_feature 1..36: note = Pin II rev primer
  source 1..36: mol_type = other DNA
gccatggatcggtaccgcaaaacacacctagactag

SEQ ID NO: 107 - DNA, 24 bp, synthetic construct
  misc_feature 1..24: note = GUS forward primer for RTPCR
  source 1..24: mol_type = other DNA
atggtccgtcctgtagaaacccca

SEQ ID NO: 108 - DNA, 24 bp, synthetic construct
  misc_feature 1..24: note = GUS reverse primer for RT-PCR
  source 1..24: mol_type = other DNA
tcattgtttgcctccctgctgcgg

SEQ ID NO: 109 - DNA, 20 bp, synthetic construct
  misc_feature 1..20: note = GUS iF primer
  source 1..20: mol_type = other DNA
caccgcgtctttgatcgcgt

SEQ ID NO: 110 - DNA, 20 bp, synthetic construct
  misc_feature 1..20: note = DsRed iR primer for amplicon B
  source 1..20: mol_type = other DNA
atgtcccaggcgaagggcag

SEQ ID NO: 111 - DNA, 20 bp, synthetic construct
  misc_feature 1..20: note = DsRed iR primer for amplicon C
  source 1..20: mol_type = other DNA
ggaagttcacgccgatgaac

SEQ ID NO: 112 - DNA, 21 bp, synthetic construct
  misc_feature 1..21: note = DsRed iR priemr for amplicon D
  source 1..21: mol_type = other DNA
ggaacaggtggtggcggccct

SEQ ID NO: 113 - DNA, 21 bp, synthetic construct
  misc_feature 1..21: note = PinII primer for amplicon E
  source 1..21: mol_type = other DNA
acacacaactttgatgcccac

SEQ ID NO: 114 - DNA, 23 bp, synthetic construct
  misc_feature 1..23: note = GUS probe
  source 1..23: mol_type = other DNA
aacgtgctgatggtgcacgacca

SEQ ID NO: 115 - DNA, 21 bp, synthetic construct
  misc_feature 1..21: note = forward primer for GUS qRTPCR
  source 1..21: mol_type = other DNA
cttacgtggcaaaggattcga

SEQ ID NO: 116 - DNA, 20 bp, synthetic construct
  misc_feature 1..20: note = reverse primer for GUS qRTPCR
  source 1..20: mol_type = other DNA
gccccaatccagtccattaa

SEQ ID NO: 117 - DNA, 21 bp, synthetic construct
  misc_feature 1..21: note = DsRed probe
  source 1..21: mol_type = other DNA
cggcgtgaacttcccctccga

SEQ ID NO: 118 - DNA, 20 bp, synthetic construct
  misc_feature 1..20: note = fwd primer DsRed qRTPCR
  source 1..20: mol_type = other DNA
aggacggctccttcatctac

SEQ ID NO: 119 - DNA, 20 bp, synthetic construct
  misc_feature 1..20: note = rev primer DsRed qRTPCR
  source 1..20: mol_type = other DNA
gtcttcttctgcattacggg

SEQ ID NO: 120 - DNA, 22 bp, synthetic construct
  misc_feature 1..22: note = MOPAT probe
  source 1..22: mol_type = other DNA
accgtgaacttccgcaccgagc

SEQ ID NO: 121 - DNA, 21 bp, synthetic construct
  misc_feature 1..21: note = fwd primer MOPAT qRTPCR
  source 1..21: mol_type = other DNA
cgtgaaccactacatcgagac

SEQ ID NO: 122 - DNA, 18 bp, synthetic construct
  misc_feature 1..18: note = rev primer MOPAT qRTPCR
  source 1..18: mol_type = other DNA
gtcgatccactcctgcgg

SEQ ID NO: 123 - DNA, 17 bp, synthetic construct
  misc_feature 1..17: note = Gr5 probe sequence
  source 1..17: mol_type = other DNA
ttgaagtcacaaagcca

SEQ ID NO: 124 - DNA, 21 bp, synthetic construct
  misc_feature 1..21: note = fwd primer Gr5 qRTPCR
  source 1..21: mol_type = other DNA
ggcagtttggttgatgctcat

SEQ ID NO: 125 - DNA, 26 bp, synthetic construct
  misc_feature 1..26: note = rev primer Gr5 qRTPCR
  source 1..26: mol_type = other DNA
tgctgtatatctttgctttgaaccat

SEQ ID NO: 126 - DNA, 39 bp, synthetic construct
  misc_feature 1..39: note = primer for polyA mapping
  source 1..39: mol_type = other DNA
gcgacacgacggcacggtttttttttttttttttttttt

SEQ ID NO: 127 - DNA, 20 bp, synthetic construct
  misc_feature 1..20: note = primer for polyA mapping
  source 1..20: mol_type = other DNA
caccgcgtctttgatcgcgt

SEQ ID NO: 128 - DNA, 20 bp, synthetic construct
  misc_feature 1..20: note = primer for polyA mapping
  source 1..20: mol_type = other DNA
gcgacacgacggcacggttt

SEQ ID NO: 129 - DNA, 313 bp, Arabidopsis thaliana
  source 1..313: mol_type = genomic DNA
taaacttgaagtctgttaaatttgatgacacctctcggtaccaatcgaagaaatctagtgaatttccgggatttttagatatctgattctgttgcgaagtttttttatccttattagtgtctgttgagtgtagattgcttggcgagtagcccacaataaagaaataaaaatggcgaatctttagctttatttactctgtttttcatcgaaacagattcagtaatatatactctgttttttcatcttacatttgtttgcaaaaccgatattatcaacttctcagtaacacaattcgttcctgaaagagtgcaaa

SEQ ID NO: 130 - DNA, 296 bp, Arabidopsis thaliana
  source 1..296: mol_type = genomic DNA
ttatggagattttaagtatcttttgatgatagtttacacactttgaataagctgattctactttatttctctctgtttgtttactgcttcagtgttttaagtcttctctttgtgtttgttgctgttgtgtctttgatggatttttgtgatccgatttaataaaagaagtgtgttcaaagtaattctctagtctctttatttgaaattccggggatagcagattggtttccgcaacattgactccaagtttataatgatcttgatgatggtctaaaagttttgaataagctgactct

SEQ ID NO: 131 - DNA, 314 bp, Arabidopsis thaliana
  source 1..314: mol_type = genomic DNA
gagatgaagaaattgctcttatggttctgaaaacttctaatatgtcgagttgttctgagttttaagattttccaaaatgtctttgtctttttttttatatctttagagttacttgaacattgtgactacttctagggttgggtttgtgtcaggtctgttatatcgtgtggtgggtctgtctaatactgattcaagtttttgttattcagctaaggaacttttcttgtttctgaacaaatcttttggttccctagagtaaaacttgacttccaaaagatagacttcctaagatcactggaatagataggagtgac

SEQ ID NO: 132 - DNA, 346 bp, Arabidopsis thaliana
  source 1..346: mol_type = genomic DNA
aactgagatggaagaagaagttgcaatagtttttttaaactagtttctttctacgtactctgttttttctctgcttggttcatggtttgagtgggcaactactgatctttcccattttcctgccggatttgtagaagaataaaaaggttgaaaatgatcatcttgagatggtgatgaaactcttaagatcctcctcttctgcttctctttttatttgccgtgtcataatgaaaattgcatattcagatgggcctgtgtgttgagtaagtggacctgtgtccagagagttgattgagatgggcttcattttaggcccacatgtttatcgatcaggttcattctcttc

SEQ ID NO: 133 - DNA, 333 bp, Arabidopsis thaliana
  source 1..333: mol_type = genomic DNA
tgcggtatacttggcatttggttttgctgtaccctttcgaaatctttatgtcgttgtttagtttgttgtattcgactatcttcttagactttgctgaaattctcaagagatttttttactcaaacatcagtccacttgtgattggcatttatatttcaaactattgccttagttacatcttcaatagtccacttatgaacgatctttagtaaattctcttattcactcgcttactaagaattaaacctaaagagaatacagaagaattccctagagtaaggatttggtgtagtgtaaaacagactcttccctctttgtattgttcagattctg

SEQ ID NO: 134 - DNA, 219 bp, Arabidopsis thaliana
  source 1..219: mol_type = genomic DNA
gttggtttcatcttattttctgcgatttttgtacttgctggatttggaatccatttgttttagctctctcgtataagattgtctcatctttgcttgttaactctatattttgaatcatcaagatatggttttgctgttaatcattgaccttcgatatttttttgccaatccgttctctctaccaacctaagaaaaaatcactaatatctcacattagag

SEQ ID NO: 135 - DNA, 243 bp, Arabidopsis thaliana
  source 1..243: mol_type = genomic DNA
taacatcaaagagcaggaagtttttaacaaggaatctggtcgagacatccatgttctggaccagacagttttttggtttagccttaaaattccaaggttgtataagaagaacacactgtttgttattctttgcggttggtaaccaatatagtaatatcagtatttcgtctcaatctctcttgttcttataataatagaatgagaaatcgaaatggaattttcatgtgaagtggattagtaaca

SEQ ID NO: 136 - DNA, 244 bp, Arabidopsis thaliana
  source 1..244: mol_type = genomic DNA
atacttttaataacactttcaatttggtttctttttcgcacttgtgcttcttctccgctttcatcttagttatggactgttcctaagcttgccatgaataaaactgtgaataataaagcttttaatgttctgtttcttactttaagtcagacaataaacttttaacacttgtgatcatgaatccttggccttcagtgtatgattgatgctcttaacacttctggtgttgtagaggaacattttt

SEQ ID NO: 137 - DNA, 277 bp, Arabidopsis thaliana
  source 1..277: mol_type = genomic DNA
tgtgaaaccaatctggatatccctgaaatttgaataaggttatgttgtgagtttatgaagttgtggtctagtgttgtattttgtttttcttaaggataatgcatctgtgtgctttgcttatctaaaatctaaataaagtgggtttctgcattgtgactttgcacaaacttcttttgttaaaatgaataagaaatcacatcaagaaaacagaacaagaccacaacgagaagacttgaaactctctgaaggtatataaacaaacagaacctagtaaatt

SEQ ID NO: 138 - DNA, 319 bp, Arabidopsis thaliana
  source 1..319: mol_type = genomic DNA
aaaagaaatgtctttaggtgacagtaagaccatggaggagttttcagctttaaattttgcttttgtaattcagattccggaatccttcataatctctatctgagtttagttttgttgttgaatcaaacatccgatttaaagttatgttcattccatcttctctccccttttcgagcatgcgagaatatgattccccctattgaacaaatcaatcaacctttaagcgataacaaattggtgttgtgaaaattattaacttcttgatctcgttttgtcagatctaaaattactatagccaagctacctttcacgtcaagtt

SEQ ID NO: 139 - DNA, 293 bp, Arabidopsis thaliana
  source 1..293: mol_type = genomic DNA
tcactcaagtcaaattgtcttggtcgtctctttatatatttttgtatcttcttattaaaaaggtaaatttgacttttaatataatggtgtgcttattgtgatttgtcattatgtttgtacctatctcttttgtgagttgtgtgatagatatactttgtattttttcttaaaagtcaatataaatttctgtttgcgagagaaaaatcgaatctttgaatagtgaactttgtggcgataaactaatataatgattaatgaagagaatgttgcgatataggacactttcatataat

SEQ ID NO: 140 - DNA, 352 bp, Arabidopsis thaliana
  source 1..352: mol_type = genomic DNA
agagatagattgattgatggacttgtgatgagtgaataaaacagggagtagcattactcattcctcccttgtgagacactttggaatcaaatcttacgttgctgctactctctttgagtcttttcttttgtttttgttacttgacacagtttttttttgtttccggtttgttgacatttagtttctcaatctaaactccagagacttattttcgctctcttttgatcagaattaggtatatctttattcggataatccttcccatccaaactgaaattgttagtaccaacattttttttatcagaacatatctcgagtactgtgttgattggattcacatatcttatgtttc

SEQ ID NO: 141 - DNA, 243 bp, Arabidopsis thaliana
  source 1..243: mol_type = genomic DNA
ttctcaaagctttgacatgtaaagataaataaatactttctgcttgatgcagtctcatgagttttgtacaaatcatgtgaacatataaatgcgctttataagtaaatgagtgtcttgttcaatgaatcatatgaaagaatttgtatgactcagaaaattggacaatgatatagaccttccaaattttgcaccctctaatgtgagatattagtgattttttcttaggttggtagagagaacgga

SEQ ID NO: 142 - DNA, 286 bp, Arabidopsis thaliana
  source 1..286: mol_type = genomic DNA
tagcactcatggaagtcaagaagctctttgagatgaggatgacagggtggtttaaaaaagtatcgctttttcatttttgttgttgttttccgttttcatttgtgatctcttagttccgtacaaaacgaattgtgaggttaattcaacttctattttaactgatagccgagaaagcttgtctagttatttacaaatgttttatcaattgtgataatgttactaatccacttcacatgaaaattccatttcgatttctcattctattattataagaacaagagagatt

SEQ ID NO: 143 - DNA, 402 bp, Oryza sativa
  source 1..402: mol_type = genomic DNA
gcattgttttgatgctgctacctataaaagtatgagcttcagatgagaatttagagagctcccctgaggcaatgggttatgactttgttatcatcttagtactactcctatctaagggacggtgtaaggctgtaatctatgtgcggtgcaatgataaggctatcctttctgcgtttgatcatgacatctcatcgctgttcgattctttctcgctggcgtctgttatgttgaatgatgcaattcttctgtaagattccgcctttaccgaaatatctctagtatcaaatttaaattggagtacggatcctctttgaacatacggatctatttgagataattttgtctgatcatcaacgagaactcggaagcgtgccaaccttgtgggtcaattgtaacgataaa

SEQ ID NO: 144 - DNA, 482 bp, Oryza sativa
  source 1..482: mol_type = genomic DNA
aggcagcagcagtatccaactagtcatgtatccagcctaaattgaaaagtcagatcatgtgtaacatggccctcaatatttcgatgttgccttgtggaaaaaatagctggtattggtactgtgttttttactccttccagctaccgacgttcagtgttagtgcacaaaatgttcatccatggtagtttgtctacattgcagtcatgtaaagttaaacattcacgatttctgtacttttgcatgtgtaactaaatgaaatggtgtgttacattcattccttcaatcaaataatccatagaaacaaactggtttgtaactattgtaactagtcgctagtgtccatggtattttcgattgtggaacataattagtttaatctgcttcagtataacttaagccggctttatcctgtgtatgcatcagcttcgctccattgaacatccaaacatcagtataagcattttgtggcgactaatcatccg

SEQ ID NO: 145 - DNA, 396 bp, Oryza sativa
  source 1..396: mol_type = genomic DNA
gagtgaggtctaggttctgttgtctgtctgtccatcatccatgtgttgactgttgagggaactcgtttcttcttttcttcacgagatgagtttttgtgtgcctgtaatactagtttgtagcaaaggctgcgttacataaggtgatgagaattgaggtaaaatgagatctgtacactaaattcattcagactgttttggcataaagaataatttggccttctgcgatttcagaagctataaattgccatctcactaaattctccttggtcctcatggcaatgcaacgacagtgtgaagcactgaagcccgtaatgctctatcaccaccatgtacaacagaaccatatatgtccatatgtacaactcgagtgttgtttgagtggccagcaaactggct

SEQ ID NO: 146 - DNA, 415 bp, Oryza sativa
  source 1..415: mol_type = genomic DNA
ggtgacggcgacgcgatcgaacaggtggtgatcgatgctgcaacgtgtgtaaatatacagcgccggctgggtcaagagatggctcgggtgacgcgcgcgcggcgtgtcctggcgttggcgccggggcattctttagtttttcatcttttcatcatctcagatggtagatacaaaacagtgtatgtatgtagctctgtttctctctatagaaccccaacaaattttgttgttgatgttgtttatcttcatatgctttgatcttgaaatcgtctaccttactactgccgatcgttgctcaaaagtgtggaagtttgaagcatcttccacgggcgttgctctactcccatctcgtttcaccacgaaatccccttcgcatagaggcatctcagccgtacatctccaaacccacgttctg

SEQ ID NO: 147 - DNA, 290 bp, Oryza sativa
  source 1..290: mol_type = genomic DNA
ccgtggcaatggctgtcatgctttggcacatactaatagcaaggtagaatggtacagctatttcattatttttgcccttgtatatttgtatcactacatgagtaaacgacgtttagttatcgatagttttgttatgagtgatgaatgatctgcatcgtactgccaatgccttgcattctcaaatggttgcacacttgcactcatacaaagttagtacactccatcatattataaattacttttttttcaagttaaatttcctgaaagtttgataaaatttatagaaaaaa

SEQ ID NO: 148 - DNA, 443 bp, Oryza sativa
  source 1..443: mol_type = genomic DNA
acaccacctgctgtctgcgcttgtttgttgccacggctgcgctacaaacacaagccgtatcatctgccgaacagctccatcgttagcttcttttgatgccttacatctatcgctggacgtttatcgtgcgtacttgtcccatacagggattgtgtttggatgatttgtagagagggtactgtatggcataagatccttcatattaatgttctgtgggtcacatatcaacttgctattattgtattcagcggttctctctctattttgccttaagctgtggtaccattttaagtccagtgcttgcttttgattgcttttctgagctttccaggacatggaatcgagcctgtgaatgtcgtcggccggtttatcctttcgactaataatttaaattgtccagtgttggttaggaatggagtatcagattctggcggtttgcatgc

SEQ ID NO: 149 - DNA, 405 bp, Oryza sativa
  source 1..405: mol_type = genomic DNA
gctgaaatttggttatctgttatgagcatatctgatttcgatacccactgttatgaaactgaataaaccgcattcctgtatgctgggaattttctcgttagtgtacgctccaatactcgtgccgttttcaaagacaagcagtgatatgcaaagcaatgctgtgtatcttgtgtgttacatcaggagttttttttaatggtgatttcgttcatagttttcagacatctgtttccgtcacacgttcgtgggaatgacttttttcatatgaaatgcgcccatggctgcgcctgaaataattttacttgtcccgtttgggatgaatgaattcttttcctgttcgttgttcagtaattcacacagagtcataaacataccggtggaaacatgctcacaaagaaagaaaca

SEQ ID NO: 150 - DNA, 431 bp, Oryza sativa
  source 1..431: mol_type = genomic DNA
agtgttacttattgttgctactatccatattcgctaccatgcttatcatcgctagattaaaggacatgtattccagtatggtcgtgctttttgcttggtactctggtcacatttgtgatgaatgttcgcagttttcgtggtcatggtctggattctacattccacctaaagaagtttttacctggttatttaatccttgagttgttgaattagctcatggtaattgtcagtagtcttccacttggttctctctgtggatgatcgttcttgttctctgtttgcgctgtggctgattgttctcatgtttttaaggtttttgggttagtcactggtcgcttcgtcactgtgagccaaaccatatgccggtagttttcttcttcaccatcatccgtttgatcctgactcgcggatctcctaatccaacttctctc

SEQ ID NO: 151 - DNA, 423 bp, Oryza sativa
  source 1..423: mol_type = genomic DNA
ttggcagcctacccactctggtaggaaaaatggaaggggcaagaagatacagtcgatgatatttgatgtgctacataccaattgtaatatttcattcatatctagagctagaaggatattcacttgtatttcagactctggttcacggtttaaattgatattgtggtagaatgctttcttggatgggtatatagtgtgactcgttaaaaatatgacaaaacggttcagctactctttacaagtggtacggcaaattgtagacccaattgcacttggcggtgagctgctttgttggagcaacagggcactgtctgcttgcgtggatgaaaagctgagtcatctgaagctagtgaacattttttcgctggctcgctgcgtgtgttttttctctgattgtggcccggttgattatgaaaatgacca

SEQ ID NO: 152 - DNA, 349 bp, Oryza sativa
  source 1..349: mol_type = genomic DNA
ggtgttccatatagatgatcagatttctataaccacatgagtacaatgtagagaattcaaatgttttgtaaccacgtgagctttgtacgctatttgcaccacagttctccctgtatgttgttggctcttttacaattaagaataatgcatgttgaagtatgagtcttcctgtttgcttctccttgatgatgaaacgctgctgcatcaactgattatttttcagagaataaacgataaacaaagcacggtgaagtatcgtgtaacccaccataagtggtagagccgtagaggtaggactgtgcatgctgaattgtaattaccatggttagttcagcagaatttgcaaaaa

SEQ ID NO: 153 - DNA, 380 bp, Oryza sativa
  source 1..380: mol_type = genomic DNA
aagaaaggaaaatgagtcctgcggattctcccttggtatgtaaatatgatggatgtgtagataaataaaatccagtttgttgttgtgtttcctgctgtgtgaggcgttaagagaacttctggcgtgctttgatttggagcagttaccatgactgttggactggtttttgtatgtattgatggacaaagctagtaagcattaatctatgagctgcagttgttttccttgatcaatgattatatatcagctgctgttgtattggttatagtggattctgactttctgataccatttcagtcttcaccaaaacagcaggctcaactacacttctcctgccatttcatcatcagaggaaaaagggggaactatattctccctgc

SEQ ID NO: 154 - DNA, 343 bp, Oryza sativa
  source 1..343: mol_type = genomic DNA
gcttcactccagttccctatcttggaaacttttgcatccgtgcaaatgtaatctctgctctcgtgttagcttgtacggagtagtgttctgattatggcaaagacgttgatgtaagcactcggcacctctttatcgatgtggattttttcccggttggtatttgaccagatgttcttgttcaagaatatgctacgttacattgtaattactgtgccatgatgctctagcttctcagtgagtggtcgaatccttccccaacttcttactaatatgcgaaatttgcattccatctcacagcatcacgtctctactcttcagttggtagaaagcattgccctatctc

SEQ ID NO: 155 - DNA, 380 bp, Oryza sativa
  source 1..380: mol_type = genomic DNA
aatgctgctatccaacaggtggagaaattcggtgaacagggtagggtatttcagaaagtttttagcatgagaacactggtggaacagagtttttgttcatctataggctctcggcggttaattccgaccatctgttatctaagctatcataaatcttgtgactcatgtctgttccaacatatgctataaatgaatgactcgctaaagatctacaagattttggtgccatttccttaccaattcctttgtgtcgttgcagtatgctgtagtgtatcggcgttgctgttcccagactcgagtgttgttgtgttctgtcttctgttgcttgggcttgttatgtatgattgagtttatagtattctctctcgtgtggcttggtc

SEQ ID NO: 156 - DNA, 466 bp, Oryza sativa
  source 1..466: mol_type = genomic DNA
tgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatc

SEQ ID NO: 157 - DNA, 482 bp, Oryza sativa
  source 1..482: mol_type = genomic DNA
atgaaccagcgtaagtactcaactgctgagatcgcatccttttggagcatcatctatcttgtcaagcttatttgcaaatagctaagccgtctctctatgtgtggtctgtaatattaaccgtgcgtaaaccagtggcaagaagtccctgtccgtgtcatggttatcgagtctgtctacttaataacgtgtgataccaggcattgtgcctgtattttaacagtaaaaattgggtacgttgtttgtgtttgtatatatattggagttggtttacctcagctggatggagtaccctacattcgcattgtactgcctacgttttatattacaagtcattttaacttttttttgaagttaaacttttatatgtttaatcaagtttatagaaaaacatagtaatatatttaacacaaaacaaaaaaaaatcaaaatatgtttaatgttagatgtaatgaaattaatttagtgttataagcgtcgttata

SEQ ID NO: 158 - DNA, 384 bp, Oryza sativa
  source 1..384: mol_type = genomic DNA
tacttgtagcaatacaaaattacagtagcattgtacactactgccgtgccagctccatgcatagtcagcagctggccactctctagctatatctagatgcgagagaattttaaggatgtaaatcatgccttcacatgaataaatcgttcgtccgtgcgttgtgtattcatgtaaattttgacggatggtcaagtaaaaataacaatggcaaattaatttagggaagaatcaaatatgtttgcgttccttttctgagccgtggatgttgttaatcagttctaccaatcgctatttgtaaaaagtttcttaaagaaatttccttcttgacatcaatcaaagtactatatataaatcttcaggataaggcacgtcgccgaccaaaca

SEQ ID NO: 159 - DNA, 445 bp, Oryza sativa
  source 1..445: mol_type = genomic DNA
tttgtctctcggccattgtgtatttttcctctcaagttgtatatgttcgccccggaggaaaagttttgtttcttctttcgctgcctcttttgtacctgtggtttccctggcgtgagagccagtctgttgcacgcctgatccctcttttatcgggagaaaggttcaacgttcgggtgctagatacaaaatgggttcctagagcccttcttggtcgtttatatttagtgtactattagtgataattggtgtaaattagttttccccagtttcctttttagaactcatgtagactttcttgcatctgatgttagtaaaattttgctctaggtgtgctggctattcttatccttagcctgacttcatgatgtttctttctttgtgagcatgtttccaccggtatgtttatgactctgtgtgaattactgaacaacgaacaggaaaagaa

SEQ ID NO: 160 - DNA, 371 bp, Oryza sativa
  source 1..371: mol_type = genomic DNA
aggccaaggcgatctgtgactatgtgatgaacaaacagagcccttccagtctgatggctcatggctgtgtgaccctgtgaatccagcattgtgtgtgcgcgcgcgcgtcatgaattgtcaaaaattatggctggaaaaccaagtggtttctggcaatatgtgttgtaccgcttgtatgtaaatgttggtaaaaacaaagtaacatcgcttatctctgaatctctaacgtaagtggttgcttaacaaaagctgctttgccccgctgtaagcctgctacactagcgtggtttcttacatttttgcctgctataaattctgttcaatttcaaggggttccaagctatgtttccaacgttgggtgagtttggatt

SEQ ID NO: 161 - DNA, 411 bp, Oryza sativa
  source 1..411: mol_type = genomic DNA
ttcatttcgtcatgcatcatatcaccctaaatatgcatgcgccaataaaaacaagtcttgattttattttgttcattgctccatcttcagtcagtgtccactggtttgtctgtcatgcatggatcatttttactcttcaattcagttctgcatttcagttgagctgttcagctgagtgccttgatttgtaccacctatctgccttgttacaaatttatgtcaaagtcttcttgctctggtacagttttacgttttcttctgcaatactagtacgcacaggaagctaagctgacaccttccaactcttcaagaaaacgaaagctgcaacaagtgtagcacgattcacgaccccgagcatgagctaggttaggcaaaacaaacaaatcagcagggtcggatgactttacttac

SEQ ID NO: 162 - DNA, 519 bp, Oryza sativa
  source 1..519: mol_type = genomic DNA
tgaaatcaatactgttggctggtgtttgcatagagaaacaatcacaattacttagtgagtcacatcctaaattctaactagaacggcacatgcccgaaattttgactgcaaattgctttgttggattgtgttacacggcttcagagcggttgccaaaagttcatttagaagaaaggctgtcagaagttatcctataaatttaaaaatagtgcgcttccaaaccaagcgcctttttcagttgttcggtacatcaaatatgtattgtataccttggaggcttggaatataaatatgggcacgctagtgctacagccatttgctactgatgtaccagcaaccattggtttgtaccttgattaattcatgacgatggtctaattctgttgtcctctttttgcaaattctgctgaactaaccatggtaattacaattcagcatgcacagtcctacctctacggctctaccacttatggtgggttacacgatacttcaccgtgctttgtttatcgtttattctct

(19) United States
(12) Patent Application Publication
(10) Pub. No.: US 2026/0258433 A1
(43) Pub. Date: Sep. 3, 2026

(54) NOVEL PLANT TERMINATOR SEQUENCES

(71) Applicant: CORTEVA AGRISCIENCE LLC, INDIANAPOLIS, IN (US)

(72) Inventors: PRIYANKA BHYRI, JOHNSTON, IA (US); NANDINI KRISHNAMURTHY, GRIMES, IA (US); ESWAR NARAYANAN, GRIMES, IA (US); AJIT NOTT, JOHNSTON, IA (US); RINKU RANJAN SARANGI, ANDHRA PRADESH (IN)

(73) Assignee: CORTEVA AGRISCIENCE LLC, INDIANAPOLIS, IN (US)

(21) Appl. No.: 19/301,407

(22) Filed: Aug. 15, 2025

Related U.S. Application Data
(62) Division of application No. 17/646,887, filed on Jan. 4, 2022, now Pat. No. 12,416,010.
Application No. 17/646,887 is a division of application No. 16/032,228, filed on Jul. 11, 2018, now Pat. No. 11,248,233.
Application No. 16/032,228 is a division of application No. 14/129,551, filed on Jul. 13, 2012, now Pat. No. 10,059,953.
(60) Provisional application No. 61/557,433, filed on Nov. 9, 2011.

(30) Foreign Application Priority Data
Jul. 15, 2011 (IN) 2001/DEL/2011

(51) Int. Cl. C12N 15/82

(52) U.S. Cl. CPC C12N 15/8216; C12N 15/8222

(57) ABSTRACT

This invention relates to gene expression regulatory sequences, specifically transcription terminator sequences. Plant transcription terminator sequences are described herein. Methods for identifying novel plant transcription terminator sequences that can terminate transcription in one orientation or in a bidirectional manner and methods of using these terminator sequences to generate transgenic plants are described herein.

### CROSS REFERENCE TO RELATED APPLICATIONS

This application claims priority to U.S. application Ser. No. 14/129,551, filed on Dec. 27, 2013, which is a 371 (National Stage) of PCT/US2012/046686, filed on Jul. 13, 2012, which claims the benefit of Indian Provisional Application No. 2001/DEL/2011, filed Jul. 15, 2011, and U.S. Provisional Application No. 61/557,433, filed Sep. 11, 2011, the entire contents of each is herein incorporated by reference.

### FIELD OF INVENTION

The present invention relates to the field of plant molecular biology and plant genetic engineering. More specifically, it relates to novel plant terminator sequences and their use to regulate gene expression in plants.

### BACKGROUND

Recent advances in plant genetic engineering have opened new doors to engineer plants to have improved characteristics or traits. These transgenic plants characteristically have recombinant DNA constructs in their genome that have a protein-coding region operably linked to multiple regulatory regions that allow accurate expression of the transgene. A few examples of regulatory elements that help regulate gene expression in transgenic plants are promoters, introns, terminators, enhancers and silencers.

Plant genetic engineering has advanced to introducing multiple traits into commercially important plants, also known as gene stacking. This can be accomplished by multigene transformation, where multiple genes are transferred to create a transgenic plant that might express a complex phenotype, or multiple phenotypes. But it is important to modulate or control the expression of each transgene optimally. The regulatory elements need to be diverse, to avoid introducing into the same transgenic plant repetitive sequences, which have been correlated with undesirable negative effects on transgene expression and stability (Peremarti et al (2010) Plant Mol Biol 73:363-378; Mette et al (1999) EMBO J 18:241-248; Mette et al (2000) EMBO J 19:5194-5201; Mourrain et al (2007) Planta 225:365-379, U.S. Pat. Nos. 7,632,982, 7,491,813, 7,674,950, PCT Application No. PCT/US2009/046968). Therefore it is important to discover and characterize novel regulatory elements that can be used to express heterologous nucleic acids in important crop species. Diverse regulatory regions can be used to control the expression of each transgene optimally.

Regulatory sequences located downstream of protein-coding regions contain signals required for transcription termination and 3′ mRNA processing, and are called terminator sequences. The terminator sequences play a key role in mRNA processing, localization, stability and translation (Proudfoot, N, (2004) Curr Opin Cell Biol 16:272-278; Gilmartin, G. M. (2005) Genes Dev. 19:2517-2521). The 3′ regulatory sequences contained in terminator sequences can affect the level of expression of a gene. Optimal expression of a chimeric gene in plant cells has been found to be dependent on the presence of appropriate 3′ sequences (Ingelbrecht et al. (1989) Plant Cell 1:671-680). Read-through transcription through a leaky terminator of a gene can cause unwanted transcription of one transgene from the promoter of another one. Also, bidirectional, convergent transcription of transgenes in transgenic plants that have leaky transcription termination of the convergent genes can lead to overlapping transcription of the convergent genes. Convergent, overlapping transcription can decrease transgene expression, or generate antisense RNA (Bieri, S. et al (2002) Molecular Breeding 10:107-117). This underlines the importance of discovering novel and efficient transcriptional terminators.

### SUMMARY

Regulatory sequences for modulating gene expression in plants are described. Specifically, regulatory sequences that are transcription terminator sequences are described. Recombinant DNA constructs comprising terminator sequences are provided.

One embodiment is a recombinant construct comprising an isolated polynucleotide comprising (a) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (b) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (c) a functional fragment of either (a) or (b); wherein the isolated polynucleotide functions as a transcriptional terminator in a plant cell. In another embodiment, the isolated polynucleotide is operably linked to the 3′ end of a heterologous polynucleotide which is operably linked to a promoter.

One embodiment is a recombinant construct comprising an isolated polynucleotide comprising (a) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (b) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (c) a functional fragment of either (a) or (b); wherein the isolated polynucleotide functions as a bidirectional transcriptional terminator in a plant cell. Another embodiment is the recombinant construct wherein the bidirectional transcriptional terminator is operably linked to (a) the 3′ end of a first heterologous polynucleotide which is operably linked to a first promoter; and (b) the 3′ end of a second heterologous polynucleotide which is operably linked to a second promoter; wherein the first and the second heterologous polynucleotides are transcribed in a convergent manner.

One embodiment is a method of expressing a heterologous polynucleotide in a plant, comprising the steps of (a) introducing into a regenerable plant cell a recombinant construct wherein the recombinant construct comprises an isolated polynucleotide comprising (i) a nucleotide sequence as set forth in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a transcriptional terminator in a plant cell and further wherein the isolated polynucleotide is operably linked to the 3′ end of a heterologous polynucleotide which is operably linked to a promoter; (b) regenerating a transgenic plant from the regenerable plant cell of (a), wherein the transgenic plant comprises in its genome the recombinant construct; and (c) obtaining a progeny plant from the transgenic plant of step (b), wherein the progeny plant comprises in its genome the recombinant DNA construct and exhibits expression of the heterologous polynucleotide.

Another embodiment provides for a method of regulating the expression of two heterologous polynucleotides in a plant, comprising the steps of: (a) introducing into a regenerable plant cell a recombinant construct, wherein the recombinant construct comprises an isolated polynucleotide comprising (i) a nucleotide sequence as set forth in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a bidirectional transcriptional terminator in a plant cell and further wherein the bidirectional transcriptional terminator is operably linked to the 3′ end of a first heterologous polynucleotide which is operably linked to a first promoter and the 3′ end of a second heterologous polynucleotide which is operably linked to a second promoter; wherein the first and the second heterologous polynucleotides are transcribed in a convergent manner; (b) regenerating a transgenic plant from the regenerable plant cell of (a), wherein the transgenic plant comprises in its genome the recombinant construct; and (c) obtaining a progeny plant from the transgenic plant of step (b), wherein the progeny plant comprises in its genome the recombinant DNA construct and exhibits expression of both the first heterologous polynucleotide and the second heterologous polynucleotide.

Another embodiment is a vector, cell, microorganism, plant, or seed comprising a recombinant DNA construct comprising a terminator sequences described herein.

Another embodiment is a regenerated, mature and fertile transgenic plants comprising the recombinant DNA constructs described above, transgenic seeds produced therefrom, T1 and subsequent generations. The transgenic plant cells, tissues, plants, and seeds may comprise at least one recombinant DNA construct of interest.

In one embodiment, the plant comprising the terminator sequences described herein is selected from the group consisting of: Arabidopsis, maize, soybean, sunflower, sorghum, canola, mustard, wheat, alfalfa, cotton, rice, barley, millet, sugar cane and switchgrass.

In one embodiment, the plant comprising the terminator sequences described herein is a monocotyledenous plant. In another embodiment, the plant comprising the terminator sequences described herein is a rice plant.

### REFERENCE TO SEQUENCE LISTING SUBMITTED ELECTRONICALLY

The official copy of the sequence listing is submitted electronically via EFS-Web as an XML formatted sequence listing with a file named 115280-US-DIV-3 Seq List.XML created on Aug. 11, 2025, and having a size of 227,000 bytes and is filed concurrently with the specification. The sequence listing comprised in this XML formatted document is part of the specification and is herein incorporated by reference in its entirety.

The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984), which are herein incorporated by reference in their entirety.

### BRIEF DESCRIPTION OF THE DRAWINGS

The invention can be more fully understood from the following detailed description and the accompanying drawings.

FIG. 1 is a schematic representation of the binary plant transformation vector, the Terminator Test Vector (TTV; PHP49597) used for testing terminators carrying the GUS reporter gene driven by the Maize Ubiquitin promoter. GUSINT is the β-glucuronidase gene with an intron inserted at SnaBI site to prevent bacterial expression. The Acc65I site used for cloning of putative terminator sequences to be tested is also shown.

FIG. 2 depicts the GUS quantitative assay of leaf samples of single-copy stable rice events harboring the constructs.

FIG. 3 shows the qRT-PCR data of single-copy stable rice events harboring TTV constructs containing the candidate terminator sequences: No terminator control (TTV), PINII terminator, T1 (SEQ ID NO:2), T4-T28 (SEQ ID NOS: 5-29), T30-T34 (SEQ ID NOS: 31-35).

SEQ ID NO: 1 is the sequence of the terminator test vector (TTV; PHP49597) carrying GUS (β-glucuronidase) reporter gene driven by the maize ubiquitin promoter.

SEQ ID NO:2-35 are the sequences of the candidate terminator sequences from Arabidopsis thaliana and Oryza sativa, as given in Table 1.

TABLE 1

| SEQ | | |
|---|---|---|
| ID | | |
| NO | Name | Species |
| 2 | T1 | Arabidopsis thaliana |
| 3 | T2 | Arabidopsis thaliana |
| 4 | T3 | Arabidopsis thaliana |
| 5 | T4 | Arabidopsis thaliana |
| 6 | T5 | Arabidopsis thaliana |
| 7 | T6 | Arabidopsis thaliana |
| 8 | T7 | Arabidopsis thaliana |
| 9 | T8 | Arabidopsis thaliana |
| 10 | T9 | Arabidopsis thaliana |
| 11 | T10 | Arabidopsis thaliana |
| 12 | T11 | Arabidopsis thaliana |
| 13 | T12 | Arabidopsis thaliana |
| 14 | T13 | Arabidopsis thaliana |
| 15 | T14 | Arabidopsis thaliana |
| 16 | T15 | Oryza sativa |
| 17 | T16 | Oryza sativa |
| 18 | T17 | Oryza sativa |
| 19 | T18 | Oryza sativa |
| 20 | T19 | Oryza sativa |
| 21 | T20 | Oryza sativa |
| 22 | T21 | Oryza sativa |
| 23 | T22 | Oryza sativa |
| 24 | T23 | Oryza sativa |
| 25 | T24 | Oryza sativa |
| 26 | T25 | Oryza sativa |
| 27 | T26 | Oryza sativa |
| 28 | T27 | Oryza sativa |
| 29 | T28 | Oryza sativa |
| 30 | T29 | Oryza sativa |
| 31 | T30 | Oryza sativa |
| 32 | T31 | Oryza sativa |
| 33 | T32 | Oryza sativa |
| 34 | T33 | Oryza sativa |
| 35 | T34 | Oryza sativa |

SEQ ID NO: 36 is the sequence of the PINII terminator.

SEQ ID NOS: 37-106 are the primers used for amplifying the candidate terminator sequences and the PINII terminator sequence, as given in Table 2.

SEQ ID NOS: 107-113 are the primer sequences used for RT-PCR to determine read through transcription for the candidate terminator sequences.

SEQ ID NOS: 114-125 are the sequences of the probes and primers used for qRT-PCR (quantitative reverse transcriptase PCR) for testing the candidate terminator sequences, as given in Table 4.

SEQ ID NOS: 126-128 are the sequences of the primers used for polyA mapping.

SEQ ID NOS: 129-162 are the sequences of the shorter terminator sequences.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot includes the Gramineae.

The terms “dicot” and “dicotyledonous plant” are used interchangeably herein. A dicot includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

Promoters that cause a gene to be expressed in most cell types at most times are commonly referred to as “constitutive promoters”.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Examples of inducible or regulated promoters include, but are not limited to, promoters regulated by light, heat, stress, flooding or drought, pathogens, phytohormones, wounding, or chemicals such as ethanol, jasmonate, salicylic acid, or safeners.

“Enhancer sequences” refer to the sequences that can increase gene expression. These sequences can be located upstream, within introns or downstream of the transcribed region. The transcribed region is comprised of the exons and the intervening introns, from the promoter to the transcription termination region. The enhancement of gene expression can be through various mechanisms which include, but are not limited to, increasing transcriptional efficiency, stabilization of mature mRNA and translational enhancement.

An “intron” is an intervening sequence in a gene that is transcribed into RNA and then excised in the process of generating the mature mRNA. The term is also used for the excised RNA sequences. An “exon” is a portion of the sequence of a gene that is transcribed and is found in the mature messenger RNA derived from the gene, and is not necessarily a part of the sequence that encodes the final gene product.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Overexpression” refers to the production of a gene product in transgenic organisms that exceeds levels of production in a null segregating (or non-transgenic) organism from the same experiment.

“Phenotype” means the detectable characteristics of a cell or organism.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

A “favorable allele” is the allele at a particular locus that confers, or contributes to, a desirable phenotype, e.g., increased cell wall digestibility, or alternatively, is an allele that allows the identification of plants with decreased cell wall digestibility that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10:107-117). As used herein, the terms “bidirectional transcriptional terminator” and “bidirectional terminator” refer to a transcription terminator sequence that has the capability of terminating transcription in both 5′ to 3′, and 3′ to 5′ orientations. A single sequence element that acts as a bidirectional transcriptional terminator can terminate transcription initiated from two convergent promoters.

The present invention encompasses functional fragments and variants of the terminator sequences disclosed herein.

A “functional fragment” herein is defined as any subset of contiguous nucleotides of the terminator sequence disclosed herein, that can perform the same, or substantially similar function as the full length promoter sequence disclosed herein. A “functional fragment” with substantially similar function to the full length terminator disclosed herein refers to a functional fragment that retains the ability to terminate transcription largely to the same level as the full-length terminator sequence. A recombinant construct comprising a heterologous polynucleotide operably linked to a “functional fragment” of the terminator sequence disclosed herein exhibits levels of heterologous polynucleotide expression substantially similar to a recombinant construct comprising a heterologous polynucleotide operably linked to the full-length terminator sequence.

A “variant”, as used herein, is the sequence of the terminator or the sequence of a functional fragment of a terminator containing changes in which one or more nucleotides of the original sequence is deleted, added, and/or substituted, while substantially maintaining terminator function. One or more base pairs can be inserted, deleted, or substituted internally to a terminator, without affecting its activity. Fragments and variants can be obtained via methods such as site-directed mutagenesis and synthetic construction.

These terminator functional fragments may comprise at least 50 contiguous nucleotides, at least 75 contiguous nucleotides, at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, at least 500 contiguous nucleotides, at least 550 contiguous nucleotides, at least 600 contiguous nucleotides, at least 650 contiguous nucleotides, at least 700 contiguous nucleotides, at least 750 contiguous nucleotides or at least 800 contiguous nucleotides of the particular terminator nucleotide sequence disclosed herein. Such fragments may be obtained by use of restriction enzymes to cleave the naturally occurring terminator nucleotide sequences disclosed herein; by synthesizing a nucleotide sequence from the naturally occurring terminator DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these terminator fragments, such as those resulting from site-directed mutagenesis, are encompassed by the compositions of the present invention.

The terms “substantially similar” and “corresponding substantially” as used herein refer to nucleic acid fragments, particularly terminator sequences, wherein changes in one or more nucleotide bases do not substantially alter the ability of the terminator to terminate transcription. These terms also refer to modifications, including deletions and variants, of the nucleic acid sequences of the instant invention by way of deletion or insertion of one or more nucleotides that do not substantially alter the functional properties of the resulting terminator relative to the initial, unmodified terminator. It is therefore understood, as those skilled in the art will appreciate, that the invention encompasses more than the specific exemplary sequences.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, WI). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

As will be evident to one of skill in the art, any heterologous polynucleotide of interest can be operably linked to the terminator sequences described in the current invention. Examples of polynucleotides of interest that can be operably linked to the terminator sequences described in this invention include, but are not limited to, polynucleotides comprising regulatory elements such as introns, enhancers, promoters, translation leader sequences, protein-coding regions from disease and insect resistance genes, genes conferring nutritional value, genes conferring yield and heterosis increase, genes that confer male and/or female sterility, antifungal, antibacterial or antiviral genes, selectable marker genes, herbicide resistance genes and the like. Likewise, the terminator sequences described in the current invention can be used to terminate transcription of any nucleic acid that controls gene expression. Examples of nucleic acids that could be used to control gene expression include, but are not limited to, antisense oligonucleotides, suppression DNA constructs, or nucleic acids encoding transcription factors.

A recombinant DNA construct (including a suppression DNA construct) of the present invention may comprise at least one regulatory sequence. In an embodiment of the present invention, the regulatory sequences disclosed herein can be operably linked to any other regulatory sequence.

Embodiments include the following:

One embodiment is a polynucleotide comprising: (i) a nucleic acid sequence of at least 90%, 91%, 92%, 93%, 94%, 95%, 96%, 97%, 98%, 99%, or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleic acid sequence of at least 90%, 91%, 92%, 93%, 94%, 95%, 96%, 97%, 98%, 99% or 100% sequence identity, based on the Clustal V method of alignment, when compared to a functional fragment of SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a full complement of the nucleic acid sequence of (i) or (ii), wherein the polynucleotide acts as a terminator in a plant cell.

One embodiment is an isolated polynucleotide comprising a nucleotide sequence, wherein the nucleotide sequence corresponds to an allele of a terminator described herein.

Recombinant DNA constructs comprising terminator sequences are also provided.

One embodiment is a recombinant construct comprising an isolated polynucleotide comprising (a) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (b) a nucleotide sequence with at least 95% sequence identity to the sequence set forth in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (c) a functional fragment of either (a) or (b); wherein the isolated polynucleotide functions as a transcriptional terminator in a plant cell. In another embodiment, the isolated polynucleotide is operably linked to the 3′ end of a heterologous polynucleotide which is operably linked to a promoter.

One embodiment is a recombinant construct comprising an isolated polynucleotide comprising (a) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (b) a nucleotide sequence with at least 95% sequence identity to the sequence set forth in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; (c) a functional fragment of either (a) or (b); wherein the isolated polynucleotide functions as a bidirectional transcriptional terminator in a plant cell. Another embodiment of the current invention is the recombinant construct wherein the bidirectional transcriptional terminator is operably linked to (a) the 3′ end of a first heterologous polynucleotide which is operably linked to a first promoter; and (b) the 3′ end of a second heterologous polynucleotide which is operably linked to a second promoter; wherein the first and the second heterologous polynucleotides are transcribed in a convergent manner.

One embodiment is a method of expressing a heterologous polynucleotide in a plant, comprising the steps of (a) introducing into a regenerable plant cell a recombinant construct wherein the recombinant construct comprises an isolated polynucleotide comprising (i) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a transcriptional terminator in a plant cell and further wherein the isolated polynucleotide is operably linked to the 3′ end of a heterologous polynucleotide which is operably linked to a promoter; (b) regenerating a transgenic plant from the regenerable plant cell of (a), wherein the transgenic plant comprises in its genome the recombinant DNA construct and exhibits expression of the heterologous polynucleotide.

One embodiment is a method of expressing a heterologous polynucleotide in a plant, comprising the steps of (a) introducing into a regenerable plant cell a recombinant construct wherein the recombinant construct comprises an isolated polynucleotide comprising (i) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a transcriptional terminator in a plant cell and further wherein the isolated polynucleotide is operably linked to the 3′ end of a heterologous polynucleotide which is operably linked to a promoter; (b) regenerating a transgenic plant from the regenerable plant cell of (a), wherein the transgenic plant comprises in its genome the recombinant construct; and (c) obtaining a progeny plant from the transgenic plant of step (b), wherein the progeny plant comprises in its genome the recombinant DNA construct and exhibits expression of the heterologous polynucleotide.

Another embodiment provides for a method of regulating the expression of two heterologous polynucleotides in a plant, comprising the steps of: (a) introducing into a regenerable plant cell a recombinant construct, wherein the recombinant construct comprises an isolated polynucleotide comprising (i) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a bidirectional transcriptional terminator in a plant cell and further wherein the bidirectional transcriptional terminator is operably linked to the 3′ end of a first heterologous polynucleotide which is operably linked to a first promoter and the 3′ end of a second heterologous polynucleotide which is operably linked to a second promoter; wherein the first and the second heterologous polynucleotides are transcribed in a convergent manner; (b) regenerating a transgenic plant from the regenerable plant cell of (a), wherein the transgenic plant comprises in its genome the recombinant DNA construct and exhibits expression of both the first heterologous polynucleotide and the second heterologous polynucleotide.

Another embodiment provides for a method of regulating the expression of two heterologous polynucleotides in a plant, comprising the steps of: (a) introducing into a regenerable plant cell a recombinant construct, wherein the recombinant construct comprises an isolated polynucleotide comprising (i) a nucleotide sequence as set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (ii) a nucleotide sequence with at least 95% sequence identity to the sequence set forth in SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 129-161 or 162; or (iii) a functional fragment of either (i) or (ii); wherein the isolated polynucleotide functions as a bidirectional transcriptional terminator in a plant cell and further wherein the bidirectional transcriptional terminator is operably linked to the 3′ end of a first heterologous polynucleotide which is operably linked to a first promoter and the 3′ end of a second heterologous polynucleotide which is operably linked to a second promoter; wherein the first and the second heterologous polynucleotides are transcribed in a convergent manner; (b) regenerating a transgenic plant from the regenerable plant cell of (a), wherein the transgenic plant comprises in its genome the recombinant construct; and (c) obtaining a progeny plant from the transgenic plant of step (b), wherein the progeny plant comprises in its genome the recombinant DNA construct and exhibits expression of both the first heterologous polynucleotide and the second heterologous polynucleotide.

Another embodiment is a vector, cell, microorganism, plant, or seed comprising a recombinant DNA construct comprising a terminator sequence described herein.

Another embodiment encompasses regenerated, mature and fertile transgenic plants comprising the recombinant DNA constructs described above, transgenic seeds produced therefrom, T1 and subsequent generations. The transgenic plant cells, tissues, plants, and seeds may comprise at least one recombinant DNA construct of interest.

In one embodiment, the plant comprising the terminator sequences described in the present invention is selected from the group consisting of: Arabidopsis, maize, soybean, sunflower, sorghum, canola, mustard, wheat, alfalfa, cotton, rice, barley, millet, sugar cane and switchgrass.

In one embodiment, the plant comprising the terminator sequences described in the present invention is a monocotyledenous plant. In another embodiment, the plant comprising the terminator sequences described in the present invention is a rice plant.

### EXAMPLES

The present invention is further illustrated in the following Examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims.

### Example 1

### Identification of Novel Terminator Sequences

The computational strategy to identify convergent gene pairs with high co-expression frequency involved the following steps:

Analysis of Arabidopsis and rice genomes for identification of convergent gene pairs.

Analysis of their transcriptomes for identification of convergent genes with high expression.

Identification of samples in which both genes from a convergent pair showed high expression.

### Identification of Candidate Arabidopsis Terminators:

For identification of convergent gene pairs, the Arabidopsis genome GFF (General File Format) file was analyzed to identify adjacent gene models in the convergent orientation with an intergenic distance ranging from 20 bp-1000 bp between them. A total of 3535 convergent gene pairs were identified and their nucleotide sequences were then retrieved. For the transcriptomics analysis, publicly available Affymetrix® array data from the Nottingham Arabidopsis Stock Center (NASC's international Affymetrix® service) were downloaded along with their metadata including sample annotations. Samples were scaled to a mean signal value of 100 and those with poor quality and no metadata were discarded. Finally, ~3000 samples were selected for this analysis. For each gene in the shortlisted convergent gene pairs, probes on the Affymetrix® array were identified and only those probes that uniquely mapped to the selected genes were selected for further analysis. Z-scores for each of the samples were calculated using median-centering; if the z-score of a probe in a given sample was greater than two, it was considered as having high expression in that sample. Finally gene pairs were shortlisted based on the criteria that both members of the gene pairs showed high expression in at least one sample. From this analysis, 89 such pairs were identified and they were further shortlisted to 24 gene pairs that showed high co-expression in at least 10 samples. Gene annotation and tissue level expression pattern were also included as additional data for the selected gene pairs. The Arabidopsis transcriptome tiling array (Salk Institute Genomic Analysis Laboratory; Yamada et al., 2003, Science, 302 (5646): 842-846) was used to independently evaluate the co-expression data of the 24 gene pairs. Based on a combination of the number of common samples in which a gene pair was showing co-expression and correlation between the expression analyses and the tiling array, 7 gene pairs were finally selected for experimental analysis. The nucleotide sequence between stop codons of each gene pair including the 3′UTRs of both genes and the intergenic region was identified and cloned for testing.

### Identification of Candidate Rice Terminators:

A similar approach was used for identifying novel terminators from rice. The entire rice genome was obtained from the MSU Rice Genome Annotation Project Database and was analyzed to identify 2892 convergent gene pairs with an intergenic distance range of 20-1000 bp. Public Affymetrix® microarray data was downloaded from the NCBI expression repository, Gene Expression Omnibus, and good quality samples were selected for analysis. For calculating high expression, the 95th percentile value of signal intensity in each sample was calculated, i.e. only 5% of the genes on the entire chip exhibited signal intensity values greater than this threshold for the given sample. For each gene pair the samples in which both genes were above the 95^{th} percentile were identified. From this analysis, 82 gene pairs were identified as showing high co-expression in at least one sample; of these, 34 gene pairs showed high co-expression in at least 10 samples and were shortlisted for further experimental analysis. Out of these, the top 10 candidate pairs based on the number of samples in which the gene pairs showed co-expression and presence of a valid gene model were selected for testing. The nucleotide sequence between stop codons of each gene pair including the 3′UTRs of both genes and the intergenic region was identified and used for testing as transcription terminators in plant cells.

### Example 2

### Amplification and Cloning of Arabidopsis and Rice Terminator Sequences

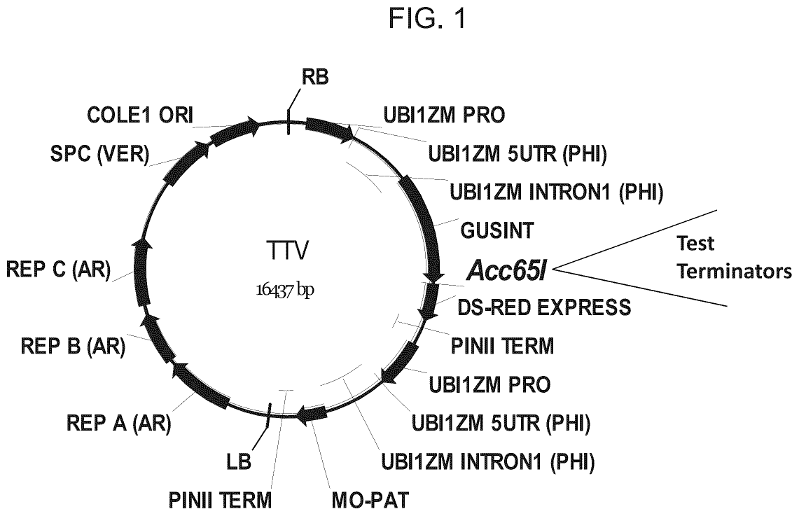

We constructed a terminator test vector (TTV) (PHP49597; FIG. 1; SEQ ID NO: 1) carrying GUS (β-glucuronidase) reporter gene driven by the maize ubiquitin promoter using standard molecular biology techniques (Sambrook et al.). A promoterless Ds-RED coding sequence was included downstream of the GUS gene for measurement of read-through transcription. The Ds-Red sequence was followed by a PinII terminator to enable termination and polyadenlylation of all read-through transcripts, so we could detect them by reverse-transcription-PCR (RT-PCR) using oligo-dT primer. The Terminator test vector also carried a monocot-optimized phosphinothricin acetyltransferase (MOPAT) gene as a plant selectable marker.

Genomic DNA was isolated from Arabidopsis thaliana and Oryza sativa leaf tissue using the QIAGEN® DNEASY® Plant Maxi Kit (QIAGEN Inc.) according to the manufacturer's instructions. Candidate terminator sequences were amplified from genomic DNA with PHUSION® DNA polymerase (New England Biolabs Inc.), using the primer sequences listed in Table 2. T1 to T14 sequences (SEQ ID NOS: 2-15) were amplified from Arabidopsis thaliana and T15 to T34 (SEQ ID NOS: 16-35) were amplified from Oryza sativa L. var. Nipponbare. T1 to T7 (SEQ ID NOS: 2-8) are complementary to sequences T8 to T14 (SEQ ID NOS: 9-15) and T15 to T24 (SEQ ID NOS: 16-25) are complementary to T25 to T34 (SEQ ID NOS: 26-35) (Table 3). The resulting DNA fragments were cloned into the terminator test vector at Acc65I restriction site using In-FUSION™ cloning (Clontech Inc.) and sequenced completely. As a positive control we cloned the potato PINII terminator (SEQ ID NO: 36; Keil et al. (1986) Nucleic Acids Res. 14:5641-5650) at the same location as the test terminators to produce the plasmid PHP49598.

All constructs were transformed into Agrobacterium (LBA4404/pSB1) and selected on spectinomycin and tetracycline. Integrity of the plasmids in Agrobacterium was confirmed by restriction digestion analysis from retransformed E. coli.

TABLE 2

| | | Amplicon | |
|---|---|---|---|
| | | Size | |
| Primer ID | Terminator | (bp) | Construct |
| TETO-1028 F (SEQ ID NO: 37) | T1 | 557 | PHP49622 |
| TETO-1029 R (SEQ ID NO: 38) | (SEQ ID NO: 2) | | |
| TETO-1207 F (SEQ ID NO: 39) | T2 | 573 | PHP51066 |
| TETO-1208 R (SEQ ID NO: 40) | (SEQ ID NO: 3) | | |
| TETO-1209 F (SEQ ID NO: 41) | T3 | 633 | PHP51067 |
| TETO-1210 R (SEQ ID NO: 42) | (SEQ ID NO: 4) | | |
| TETO-1211 F (SEQ ID NO: 43) | T4 | 639 | PHP51068 |
| TETO-1212 R (SEQ ID NO: 44) | (SEQ ID NO: 5) | | |
| TETO-1213 F (SEQ ID NO: 45) | T5 | 685 | PHP51069 |
| TETO-1214 R (SEQ ID NO: 46) | (SEQ ID NO: 6) | | |
| TETO-1215 F (SEQ ID NO: 47) | T6 | 411 | PHP51070 |
| TETO-1216 R (SEQ ID NO: 48) | (SEQ ID NO: T7) | | |
| TETO-1030 F (SEQ ID NO: 49) | T7 | 457 | PHP49623 |
| TETO-1031 R (SEQ ID NO: 50) | (SEQ ID NO: 8) | | |
| TETO-1032 F (SEQ ID NO: 51) | T8 | 557 | PHP49624 |
| TETO-1033 R (SEQ ID NO: 52) | (SEQ ID NO: 9) | | |
| TETO-1034 F (SEQ ID NO: 53) | T9 | 573 | PHP49625 |
| TETO-1035 R (SEQ ID NO: 54) | (SEQ ID NO: 10) | | |
| TETO-1217 F (SEQ ID NO: 55) | T10 | 633 | PHP51071 |
| TETO-1218 R (SEQ ID NO: 56) | (SEQ ID NO: 11) | | |
| TETO-1219 F (SEQ ID NO: 57) | T11 | 639 | PHP51072 |
| TETO-1220 R (SEQ ID NO: 58) | (SEQ ID NO: 12) | | |
| TETO-1036 F (SEQ ID NO: 59) | T12 | 685 | PHP49626 |
| TETO-1037 R (SEQ ID NO: 60) | (SEQ ID NO: 13) | | |
| TETO-1038 F (SEQ ID NO: 61) | T13 | 411 | PHP49627 |
| TETO-1039 R (SEQ ID NO: 62) | (SEQ ID NO: 14) | | |
| TETO-1040 F (SEQ ID NO: 63) | T14 | 457 | PHP49628 |
| TETO-1041 R (SEQ ID NO: 64) | (SEQ ID NO: 15) | | |
| TETO-986 F (SEQ ID NO: 65) | T15 | 782 | PHP51073 |
| TETO-987 R (SEQ ID NO: 66) | (SEQ ID NO: 16) | | |
| TETO-988 F (SEQ ID NO: 67) | T16 | 825 | PHP51074 |
| TETO-989 R (SEQ ID NO: 68) | (SEQ ID NO: 17) | | |
| TETO-990 F (SEQ ID NO: 69) | T17 | 776 | PHP51075 |
| TETO-991 R (SEQ ID NO: 70) | (SEQ ID NO: 18) | | |
| TETO-992 F (SEQ ID NO: 71) | T18 | 881 | PHP51076 |
| TETO-993 R (SEQ ID NO: 72) | (SEQ ID NO: 19) | | |
| TETO-994 F (SEQ ID NO: 73) | T19 | 772 | PHP51077 |
| TETO-995 R (SEQ ID NO: 74) | (SEQ ID NO: 20) | | |
| TETO-996 F (SEQ ID NO: 75) | T20 | 827 | PHP51078 |
| TETO-997 R (SEQ ID NO: 76) | (SEQ ID NO: 21) | | |
| TETO-998 F (SEQ ID NO: 77) | T21 | 770 | PHP51079 |
| TETO-999 R (SEQ ID NO: 78) | (SEQ ID NO: 22) | | |
| TETO-1000 F (SEQ ID NO: 79) | T22 | 814 | PHP51080 |
| TETO-1001 R (SEQ ID NO: 80) | (SEQ ID NO: 23) | | |
| TETO-1002 F (SEQ ID NO: 81) | T23 | 834 | PHP51081 |
| TETO-1003 R (SEQ ID NO: 82) | (SEQ ID NO: 24) | | |
| TETO-1004 F (SEQ ID NO: 83) | T24 | 740 | PHP51082 |
| TETO-1005 R (SEQ ID NO: 84) | (SEQ ID NO: 25) | | |
| TETO-1006 F (SEQ ID NO: 85) | T25 | 782 | PHP51083 |
| TETO-1007 R (SEQ ID NO: 86) | (SEQ ID NO: 26) | | |
| TETO-1008 F (SEQ ID NO: 87) | T26 | 825 | PHP51084 |
| TETO-1009 R (SEQ ID NO: 88) | (SEQ ID NO: 27) | | |
| TETO-1010 F (SEQ ID NO: 89) | T27 | 776 | PHP51085 |
| TETO-1011 R (SEQ ID NO: 90) | (SEQ ID NO: 28) | | |
| TETO-1012 F (SEQ ID NO: 91) | T28 | 881 | PHP51086 |
| TETO-1013 R (SEQ ID NO: 92) | (SEQ ID NO: 29) | | |
| TETO-1014 F (SEQ ID NO: 93) | T29 | 772 | |
| TETO-1015 R (SEQ ID NO: 94) | (SEQ ID NO: 30) | | |
| TETO-1016 F (SEQ ID NO: 95) | T30 | 827 | PHP51088 |
| TETO-1017 R (SEQ ID NO: 96) | (SEQ ID NO: 31) | | |
| TETO-1018 F (SEQ ID NO: 97) | T31 | 770 | PHP51089 |
| TETO-1019 R (SEQ ID NO: 98) | (SEQ ID NO: 32) | | |
| TETO-1020 F (SEQ ID NO: 99) | T32 | 814 | |
| TETO-1021 R (SEQ ID NO: 100) | (SEQ ID NO: 33) | | |
| TETO-1022 F (SEQ ID NO: 101) | T33 | 834 | |
| TETO-1023 R (SEQ ID NO: 102) | (SEQ ID NO: 34) | | |
| TETO-1024 F (SEQ ID NO: 103) | T34 | 740 | PHP51092 |
| TETO-1025 R (SEQ ID NO: 104) | (SEQ ID NO: 35) | | |
| TETO-420 F (SEQ ID NO: 105) | Pin II | 330 | PHP49598 |
| TETO-421 R (SEQ ID NO: 106) | (SEQ ID NO: 36) | | |

TABLE 3
Terminator Sequences in Inverse Orientations

| Orientation 1 | Orientation 2 | Species |
|---|---|---|
| T1 (SEQ ID NO: 2) | T8 (SEQ ID NO: 9) | Arabidopsis thaliana |
| T2 (SEQ ID NO: 3) | T9 (SEQ ID NO: 10) | Arabidopsis thaliana |
| T3 (SEQ ID NO: 4) | T10 (SEQ ID NO: 11) | Arabidopsis thaliana |
| T4 (SEQ ID NO: 5) | T11 (SEQ ID NO: 12) | Arabidopsis thaliana |
| T5 (SEQ ID NO: 6) | T12 (SEQ ID NO: 13) | Arabidopsis thaliana |
| T6 (SEQ ID NO: 7) | T13 (SEQ ID NO: 14) | Arabidopsis thaliana |
| T7 (SEQ ID NO: 8) | T14 (SEQ ID NO: 15) | Arabidopsis thaliana |
| T15 (SEQ ID NO: 16) | T25 (SEQ ID NO: 26) | Oryza sativa |
| T16 (SEQ ID NO: 17) | T26 (SEQ ID NO: 27) | Oryza sativa |
| T17 (SEQ ID NO: 18) | T27 (SEQ ID NO: 28) | Oryza sativa |
| T18 (SEQ ID NO: 19) | T28 (SEQ ID NO: 29) | Oryza sativa |
| T19 (SEQ ID NO: 20) | T29 (SEQ ID NO: 30) | Oryza sativa |
| T20 (SEQ ID NO: 21) | T30 (SEQ ID NO: 31) | Oryza sativa |
| T21 (SEQ ID NO: 22) | T31 (SEQ ID NO: 32) | Oryza sativa |
| T22 (SEQ ID NO: 23) | T32 (SEQ ID NO: 33) | Oryza sativa |
| T23 (SEQ ID NO: 24) | T33 (SEQ ID NO: 34) | Oryza sativa |
| T24 (SEQ ID NO: 25) | T34 (SEQ ID NO: 35) | Oryza sativa |

### Example 3

Rice Transformation with Candidate Terminator Sequences

The candidate terminator sequences T1-T34 (SEQ ID NOS: 2-35) can be transformed into rice plants by Agrobacterium-mediated transformation by using Agrobacterium containing the constructs described in Table 2.

### Transformation and Regeneration of Rice Callus Via Agrobacterium Infection:

O. sativa spp. japonica rice var. Nipponbare seeds are sterilized in absolute ethanol for 10 minutes then washed 3 times with water and incubated in 70% Sodium hypochlorite [Fisher Scientific-27908] for 30 minutes. The seeds are then washed 5 times with water and dried completely. The dried seeds are inoculated into NB-CL media [CHU(N6) basal salts (PhytoTechnology-C416) 4 g/l; Eriksson's vitamin solution (1000× PhytoTechnology-E330) 1 ml/l; Thiamine HCl (Sigma-T4625) 0.5 mg/l; 2,4-Dichloro phenoxyacetic acid (Sigma-D7299) 2.5 mg/l; BAP (Sigma-B3408) 0.1 mg/l; L-Proline (PhytoTechnology-P698) 2.5 g/l; Casein acid hydrolysate vitamin free (Sigma-C7970) 0.3 g/l; Myo-inositol (Sigma-13011) 0.1 g/l; Sucrose (Sigma-S5390) 30 g/l; GELRITE® (Sigma-G1101.5000) 3 g/l; pH 5.8) and kept at 28° C. in dark for callus proliferation.

A single Agrobacterium colony containing a desired insert with the candidate terminator sequences (SEQ ID NOS: 2-35) or PINII terminator (SEQ ID NO:36) from a freshly streaked plate can be inoculated in YEB liquid media [Yeast extract (BD Difco-212750) 1 g/l; Peptone (BD Difco-211677) 5 g/l; Beef extract (Amresco-0114) 5 g/l; Sucrose (Sigma-S5390) 5 g/l; Magnesium Sulfate (Sigma-M8150) 0.3 g/l at pH-7.0] supplemented with Tetracycline (Sigma-T3383) 5 mg/l, Rifamysin 10 mg/l and Spectinomycin (Sigma-5650) 50 mg/l. The cultures are grown overnight at 28° C. in dark with continuous shaking at 220 rpm. The following day the cultures are adjusted to 0.5 Absorbance at 550 nm in PHI-A (CHU(N6) basal salts (PhytoTechnology-C416) 4 g/l; Eriksson's vitamin solution (1000× PhytoTechnology-E330) 1 ml/l; Thiamine HCl (Sigma-T4625) 0.5 mg/l; 2,4-Dichloro phenoxyacetic acid (Sigma-D7299) 2.5 mg/l, L-Proline (PhytoTechnology-P698) 0.69 mg/l; Sucrose (Sigma-S5390) 68.5 g/l; Glucose-36 g/l (Sigma-G8270); pH 5.8);) media supplemented with 200 UM Acetosyringone (Sigma-D134406) and incubated for 1 hour at 28° C. with continuous shaking at 220 rpm.

17-21 day old proliferating calli are transferred to a sterile culture flask and Agrobacterium solution prepared as described above was added to the flask. The suspension is incubated for 20 minutes with gentle shaking every 2 minutes. The Agrobacterium suspension is decanted carefully and the calli are placed on WHATMAN® filter paper No-4. The calli are immediately transferred to NB-CC medium [NB-CL supplemented with 200 μM Acetosyringone (Sigma-D134406) and incubated at 21° C. for 72 hrs.

### Culture Termination and Selection:

The co-cultivated calli are placed in a dry, sterile, culture flask and washed with 1 liter of sterile distilled water containing Cefotaxime (Duchefa-C0111.0025) 0.250 g/l and Carbenicillin (Sigma-C0109.0025) 0.4 g/l. The washes are repeated 4 times or until the solution appeared clear. The water is decanted carefully and the calli are placed on WHATMAN® filter paper No-4 and dried for 30 minutes at room temperature. The dried calli are transferred to NB-RS medium [NB-CL supplemented with Cefotaxime (Duchefa-C0111.0025) 0.25 g/l; and Carbenicillin (Sigma-C0109.0025) 0.4 g/l and incubated at 28° C. for 4 days.

The calli are then transferred to NB-SB media [NB-RS supplemented with Bialaphos (Meiji Seika K.K., Tokyo, Japan) 5 mg/l and incubated at 28° C. and subcultured into fresh medium every 14 days. After 40-45 days on selection, proliferating, Bialaphos-resistant callus events are easily observable.

Regeneration of Stably Transformed Rice Plants from Transformed Rice Calli:

Transformed callus events are transferred to NB-RG media [CHU(N6) basal salts (PhytoTechnology-C416) 4 g/l; N6 vitamins 1000×1 ml {Glycine (Sigma-47126) 2 g/l; Thiamine HCl (Sigma-T4625) 1 g/l; acid; Kinetin (Sigma-K0753) 0.5 mg/l; Casein acid hydrolysate vitamin free (Sigma-C7970) 0.5 g/l; Sucrose (Sigma-S5390) 20 g/l; Sorbitol (Sigma-S1876) 30 g/l, pH was adjusted to 5.8 and 4 g/l GELRITE® (Sigma-G1101.5000) was added. Post-sterilization 0.1 ml/l of CuSo4 (100 mM concentration, Sigma-C8027) and 100 ml/l 10× AA Amino acids pH free {Glycine (Sigma-G7126) 75 mg/l; L-Aspartic acid (Sigma-A9256) 2.66 g/l; L-Arginine (Sigma-A5006) 1.74 g/l; L-Glutamine (Sigma-G3126) 8.76 g/l} and incubated at 32° C. in light. After 15-20 days, regenerating plantlets can be transferred to magenta boxes or tubes containing NB-RT media [MS basal salts (PhytoTechnology-M524) 4.33 g/L; B5 vitamins 1 ml/l from 1000× stock {Nicotinic acid (Sigma-G7126) 1 g/l, Thiamine HCl (Sigma-T4625) 10 g/l)}; Myo-inositol (Sigma-13011) 0.1 g/l; Sucrose (Sigma-S5390) 30 g/l; and IBA (Sigma-15386) 0.2 mg/l; pH adjusted to 5.8]. Rooted plants obtained after 10-15 days can be hardened in liquid Y media [1.25 ml each of stocks A-F and water sufficient to make 1000 ml. Composition of individual stock solutions: Stock (A) Ammonium Nitrate (HIMEDIA-RM5657) 9.14 g/l, (B) Sodium hydrogen Phosphate (HIMEDIA-58282) 4.03 g, (C) Potassium Sulphate (HIMEDIA-29658-4B) 7.14 g, (D) Calcium Chloride (HIMEDIA-C5080) 8.86 g, (E) Magnesium Sulphate (HIMEDIA-RM683) 3.24 g, (F) (Trace elements) Magnesium chloride tetra hydrate (HIMEDIA-10149) 15 mg, Ammonium Molybdate (HIMEDIA-271974B) 6.74 mg/l, Boric acid (Sigma-136768) 9.34 g/l, Zinc sulphate heptahydrate (HiMedia-RM695) 0.35 mg/l, Copper Sulphate heptahydrate (HIMEDIA-C8027) 0.31 mg/l, Ferric chloride hexahydrate (Sigma-236489) 0.77 mg/l, Citric acid monohydrate (HIMEDIA-C4540) 0.119 g/l] at 28° C. for 10-15 days before transferring to greenhouse. Leaf samples are collected for histochemical GUS staining with 5-bromo-4-chloro-3-indolyl-β-D-glucuronide (X-Gluc), using standard protocols (Janssen and Gardner, Plant Mol. Biol. (1989) 14:61-72).

Transgenic plants are analyzed for copy number by southern blotting using standard procedure. All single copy events are transferred to individual pots and further analysis is performed only on these. For all the analysis leaf material from three independent one month old single copy T₀ events are taken.

### Example 4

Rice Transformation with Candidate Rice Terminator Sequences

The candidate rice terminator sequences (SEQ ID NOS: 16-35) were tested for their efficacy to function as transcription terminators by transformation into rice plants by Agrobacterium-mediated transformation as described in Example 3. The constructs for generating the transgenic plants are described in Table 2.

### Example 5A

### Assays for Testing of Candidate Rice Terminator Sequences in Stably

### Transformed Rice Tissues

ReverseTranscriptase-PCR (RT-PCR) as well as quantitative RT-PCR (qRT-PCR) can be done from stably transformed rice plant tissues, to test the ability of candidate terminator sequences to stop transcription (i.e., prevent read-through transcription). QRT-PCR is the preferred way of testing the candidate terminator sequences. SEQ ID NOS: 100-113 can be used for doing RT-PCR to determine read-through transcription from the candidate terminator sequences.

### Histochemical and Fluorometric GUS Analysis:

Leaf samples from each construct can be used for histochemical GUS staining with 5-bromo-4-chloro-3-indolyl-β-D-glucuronide (X-Gluc), using standard protocols (Janssen and Gardner, Plant Mol. Biol. (1989) 14:61-72,) and three pools of leaf samples from three independent single copy events per construct may be used for quantitative MUG assay using standard protocols (Jefferson, R. A., Nature. 342, 837-8 (1989); Jefferson, R. A., Kavanagh, T. A. & Bevan, M. W. EMBO J. 6, 3901-3907 (1987).

### Example 5B

### Testing of Candidate Rice Terminator Sequences in Stably Transformed Rice Tissues

GUS Fluorometric Analysis of Rice Tissues Stably Transformed with Candidate Rice Terminator Sequences:

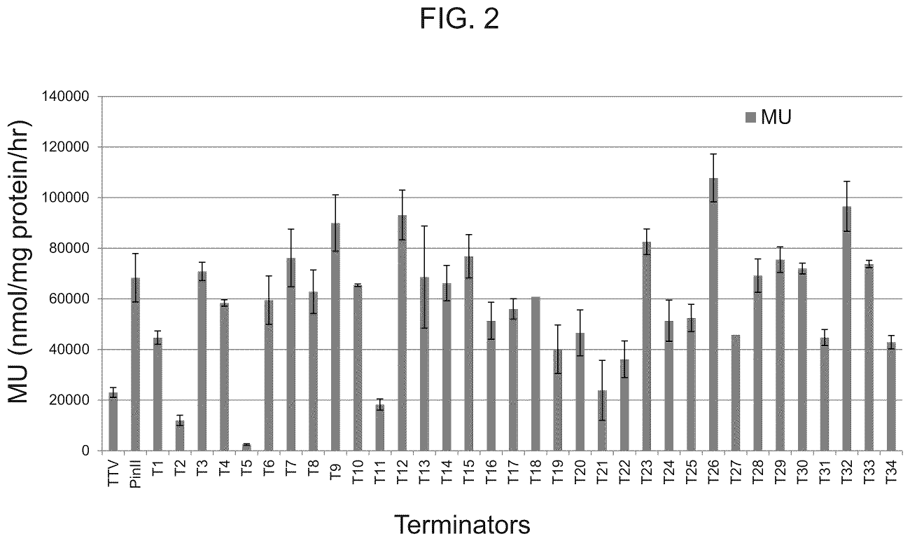

When compared with TTV, we observed higher GUS protein expression with PINII as well as test sequences T15, T16, T17, T18, T19, T20, T22, T23, T24, T25, T26, T27, T28, T29, T30, T31, T32, T33 and T34 (SEQ ID NOS: 16-21, 23-35 respectively). However, T21 (SEQ ID NO:22) had the same level of GUS expression as TTV (FIG. 2).

Quantitative Reverse Transcriptase PCR (qRT-PCR) to Determine Read Through Transcription Through Test Terminator:

qRT-PCR was performed with leaf tissue from stable transformants. The stably transformed plants were tested for the presence of read-through transcript that had passed through the PINII terminator and the test terminators. To assess presence of products that would indicate that transcription was continuing past the terminator, amplification was targeted downstream of the terminator being tested. A primer set was designed downstream of the PINII or test terminators, in the filler sequence (Ds Red). The read-through can be measured by the ratio of DsRed to GUS.

At least three pools of leaf samples from three independent single copy events were tested for each construct. The primers and probes are listed in Table 4.

TABLE 4

| Probe | Primer Sequence | | qPCR Assay |
|---|---|---|---|
| (SEQ ID NO) | (SEQ ID NO) | Fluor | Type |
| GUS | GUSFwd primer | FAM | TAQMAN ® |
| (SEQ ID NO: | (SEQ ID NO: 115) | | |
| 114) | GUS Rev primer | | |
| | (SEQ ID NO: 116) | | |
| DsRed | DsRed Fwd primer | FAM | TAQMAN ® |
| (SEQ ID NO: | (SEQ ID NO: 118) | | |
| 117) | DsRed Rev primer | | |
| | (SEQ ID NO: 119) | | |

Read-Through Transcription from Candidate Rice Sequences:

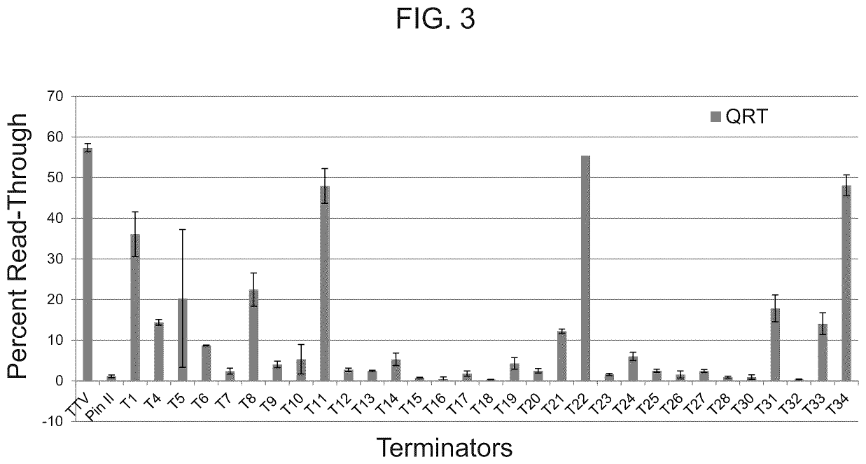

As expected, read-through transcription was observed in the terminator test vector (TTV (SEQ ID NO:1; PHP49597) as depicted in FIG. 3. The PHP49598 construct with the PINII terminator (SEQ ID NO:36) reduced the transcription read-through significantly (FIG. 3). Candidate terminator sequences from Oryza sativa T15, T16, T17, T18, T20, T23, T25, T26, T27, T28, T30 and T32 (SEQ ID NOS: 16-19, 21, 24, 26-29, 31 and 33 respectively) were able to terminate transcription efficiently as evidenced by the very low level of read-through transcripts (FIG. 3), comparable to the PINII terminator. As can be seen from Table 3, T14 and T25 (SEQ ID NOS: 15 and 26 respectively); T16 and T26 (SEQ ID NOS: 17 and 27 respectively); T17 and T27 (SEQ ID NOS: 18 and 28 respectively); T18 and T28 (SEQ ID NOS: 19 and 29 respectively); T20 and T30 (SEQ ID NOS: 21 and 31 respectively) are the same nucleotide sequence but cloned in inverted orientation. Hence these can function as bi-directional terminator sequences. Candidate terminator sequences T19, T21 and T24 (SEQ ID NOS: 20, 22 and 25 respectively) also showed less read through compared to the TTV terminator (FIG. 3)

### Example 6

Rice Transformation with Candidate Arabidopsis Terminator Sequences

The candidate Arabidopsis terminator sequences (SEQ ID NOS: 2-15) can be transformed into rice plants by Agrobacterium-mediated transformation as described in Example 3, to test their efficacy to function as transcription terminators. The constructs are described in Table 2.

### Example 7

### Testing of Candidate Arabidopsis Terminator Sequences in Stably Transformed Rice Tissues

QRT-PCR was done from stably transformed rice plant tissues, to test the ability of candidate Arabidopsis terminator sequences (SEQ ID NOS: 2-15) to stop transcription (that is prevent transcription read-through transcription) and to compare GUS expression as compared to that with PINII terminator, as described in Example 5.

Read-Through Transcription from Candidate Arabidopsis Sequences:

Terminator sequences from Arabidopsis thaliana T7, T9, T10, T12, T13, (SEQ ID NOS: 8, 10, 11, 13 and 14 respectively) were able to terminate transcription efficiently as evidenced by the very low level of read-through transcripts (FIG. 3), comparable to the PINII terminator. Terminator sequences T4, T5 and T6 also showed less read through compared to the TTV terminator (FIG. 3).

GUS Fluorometric Analysis of Rice Tissues Stably Transformed with Candidate Arabidopsis Terminator Sequences:

When compared with TTV, we observed higher GUS protein expression with PINII as well as test sequences T1, T3, T4, T6, T7, T8, T9, T10, T12, T13, and T14 (SEQ ID NOS: 2, 4, 5, 7, 8, 10, 13, 14 and 15 respectively). However, T2, T5 and T11 (SEQ ID NOS: 3, 6 and 12) had the same level of GUS expression as TTV (FIG. 2).

### Example 8

### Identification of Shorter Terminator Sequences

Each candidate bidirectional transcriptional terminator might be comprised of two convergent constituent transcriptional terminators. To identify these constituent terminator sequences, polyadenylation sites were mapped as described below.

### Mapping Polyadenylation Sites in Terminator Sequences

RNA was extracted from leaf tissue of T₀ single copy event for each construct. cDNA was synthesized using SuperScript® III First-Strand Synthesis System from INVITROGEN™ using adapter ligated oligodT primer (TETO-1527; SEQ ID NO: 126) and PCR was performed with GUS internal primer (TET0-1172; SEQ ID NO:127) and adapter reverse primers (TETO-1528; SEQ ID NO:128). The amplified products were cloned using Zero Blunt® TOPO® PCR cloning kit (INVITROGEN™). For each terminator, 40 clones were sequenced. The sequence analysis revealed multiple polyA sites. The sequences of the shorter terminator sequences corresponding to the longer terminator sequences are given in SEQ ID NOS: 129-162 and in Table 6.

TABLE 5

| | SEQ | |
|---|---|---|
| | ID | |
| Primer Name | NO | Primer ID |
| TETO-1527 | 126 | Adap-dT |
| TETO-1172 | 127 | GUS iF |
| TETO-1528 | 128 | Adap R |

TABLE 6

| Orientation 1 | Orientation 2 | Species | 5′ terminator | 3′ terminator |
|---|---|---|---|---|
| T1 (SEQ ID | T8 (SEQ ID | Arabidopsis | T1s (SEQ ID | T8s (SEQ ID |
| NO: 2) | NO: 9) | thaliana | NO: 129) | NO: 136) |
| T2 (SEQ ID | T9 (SEQ ID | Arabidopsis | T2s (SEQ ID | T9s (SEQ ID |
| NO: 3) | NO: 10) | thaliana | NO: 130) | NO: 137) |
| T3 (SEQ ID | T10 (SEQ ID | Arabidopsis | T3s (SEQ ID | T10s (SEQ ID |
| NO: 4) | NO: 11) | thaliana | NO: 131) | NO: 138) |
| T4 (SEQ ID | T11 (SEQ ID | Arabidopsis | T4s (SEQ ID | T11s (SEQ ID |
| NO: 5) | NO: 12) | thaliana | NO: 132) | NO: 139) |
| T5 (SEQ ID | T12 (SEQ ID | Arabidopsis | T5s (SEQ ID | T12s (SEQ ID |
| NO: 6) | NO: 13) | thaliana | NO: 133) | NO: 140) |
| T6 (SEQ ID | T13 (SEQ ID | Arabidopsis | T6s (SEQ ID | T13s (SEQ ID |
| NO: 7) | NO: 14) | thaliana | NO: 134) | NO: 141) |
| T7 (SEQ ID | T14 (SEQ ID | Arabidopsis | T7s (SEQ ID | T14s (SEQ ID |
| NO: 8) | NO: 15) | thaliana | NO: 135) | NO: 142) |
| T15 (SEQ ID | T25 (SEQ ID | Oryza sativa | T15s (SEQ | T25s (SEQ ID |
| NO: 16) | NO: 26) | | ID NO: 143) | NO: 153) |
| T16 (SEQ ID | T26 (SEQ ID | Oryza sativa | T16s (SEQ ID | T26s (SEQ ID |
| NO: 17) | NO: 27) | | NO: 144) | NO: 154) |
| T17 (SEQ ID | T27 (SEQ ID | Oryza sativa | T17s (SEQ ID | T27s (SEQ ID |
| NO: 18) | NO: 28) | | NO: 145) | NO: 155) |
| T18 (SEQ ID | T28 (SEQ ID | Oryza sativa | T18s (SEQ ID | T28s (SEQ ID |
| NO: 19) | NO: 29) | | NO: 146) | NO: 156) |
| T19 (SEQ ID | T29 (SEQ ID | Oryza sativa | T19s (SEQ ID | T29 (SEQ ID |
| NO: 20) | NO: 30) | | NO: 147) | NO: 157) |
| T20 (SEQ ID | T30 (SEQ ID | Oryza sativa | T20s (SEQ ID | T30 (SEQ ID |
| NO: 21) | NO: 31) | | NO: 148) | NO: 158) |
| T21 (SEQ ID | T31 (SEQ ID | Oryza sativa | T21s (SEQ ID | T31s (SEQ ID |
| NO: 22) | NO: 32) | | NO: 149) | NO: 159) |
| T22 (SEQ ID | T32 (SEQ ID | Oryza sativa | T22s (SEQ ID | T32s (SEQ ID |
| NO: 23) | NO: 33) | | NO: 150) | NO: 160) |
| T23 (SEQ ID | T33 (SEQ ID | Oryza sativa | T23s (SEQ ID | T33s (SEQ ID |
| NO: 24) | NO: 34) | | NO: 151) | NO: 161) |
| T24 (SEQ ID | T34 (SEQ ID | Oryza sativa | T24s (SEQ ID | T34s (SEQ ID |
| NO: 25) | NO: 35) | | NO: 152) | NO: 162) |

### Example 9

### Testing of Truncated Terminator Sequences in Stably Transformed Arabidopsis Tissue

Based on the polyA data obtained as described in Example 8, the terminators can be truncated and cloned as described in the Example 2.

The truncated terminators (SEQ ID NOS: 129-162) can be transformed into Arabidopsis thaliana by floral dip method (Kim J Y et al (2003) Development 130: 4351-4362). QRT-PCR and MUG analysis can be done to test the efficiency of the truncated terminators in T1 Arabidopsis leaf tissue as described in Example 5.

1. A recombinant construct comprising a polynucleotide comprising:
(a) a nucleotide sequence as set forth in SEQ ID NOS: 2, 4, 5, 6, 7, 8, 9, 11, 12, 13, 14, 15, 16, 17, 18, 19, 20, 21, 22, 23, 24, 25, 26, 27, 30, 31, 32, 33, 34, 35, 129-161 or 162; or
(b) a nucleotide sequence with at least 95% sequence identity to the sequence set forth in SEQ ID NOS: 2, 4, 5, 6, 7, 8, 9, 11, 12, 13, 14, 15, 16, 17, 18, 19, 20, 21, 22, 23, 24, 25, 26, 27, 28, 30, 31, 32, 33, 34, 35, 129-161 or 162; or
(c) a functional fragment of either (a) or (b);
wherein the polynucleotide is operably linked to the 3′ end of a heterologous polynucleotide which is operably linked to a promoter; and further wherein the polynucleotide functions as a transcriptional terminator in a plant cell.

2. The recombinant construct of claim 1 wherein the polynucleotide functions as a bidirectional transcriptional terminator.

3. (canceled)

4. The recombinant construct of claim 2 wherein the bidirectional transcriptional terminator is operably linked to:
(a) the 3′ end of a first heterologous polynucleotide which is operably linked to a first promoter; and
(b) the 3′ end of a second heterologous polynucleotide which is operably linked to a second promoter;
wherein the first and the second heterologous polynucleotides are transcribed in a convergent manner.

5. A plant comprising in its genome the recombinant construct of claim 1.

6. A seed from the plant of claim 5, wherein the seed comprises said recombinant construct.

7. The plant of claim 5, wherein said plant is selected from the group consisting of: Arabidopsis, maize, soybean, sunflower, sorghum, canola, mustard, wheat, alfalfa, cotton, rice, barley, millet, sugar cane and switchgrass.

8. The seed of claim 6, wherein said seed is selected from the group consisting of: Arabidopsis, maize, soybean, sunflower, sorghum, canola, mustard, wheat, alfalfa, cotton, rice, barley, millet, sugar cane and switchgrass.

9. A method of expressing a heterologous polynucleotide in a plant, comprising the steps of:
(a) introducing into a regenerable plant cell the recombinant construct of claim 1;
(b) regenerating a transgenic plant from the regenerable plant cell of (a), wherein the transgenic plant comprises in its genome the recombinant construct of claim 1; and
(c) obtaining a progeny plant from the transgenic plant of step (b), wherein the progeny plant comprises in its genome the recombinant construct of claim 1 and exhibits expression of the heterologous polynucleotide.

10. A method of regulating the expression of two heterologous polynucleotides in a plant, comprising the steps of:
(a) introducing into a regenerable plant cell the recombinant construct of claim 4;
(b) regenerating a transgenic plant from the regenerable plant cell of (a), wherein the transgenic plant comprises in its genome the recombinant construct of claim 4; and
(c) obtaining a progeny plant from the transgenic plant of step (b), wherein the progeny plant comprises in its genome the recombinant construct of claim 4 and exhibits expression of both the first heterologous polynucleotide and the second heterologous polynucleotide.

11. The method of claim 9, wherein said plant is selected from the group consisting of: Arabidopsis, maize, soybean, sunflower, sorghum, canola, mustard, wheat, alfalfa, cotton, rice, barley, millet, sugar cane and switchgrass.

12. The method of claim 10, wherein said plant is selected from the group consisting of: Arabidopsis, maize, soybean, sunflower, sorghum, canola, mustard, wheat, alfalfa, cotton, rice, barley, millet, sugar cane and switchgrass.

13. A plant comprising in its genome the recombinant construct of claim 4.

14. A seed from the plant of claim 13, wherein the seed comprises said recombinant construct.

15. The plant of claim 13, wherein said plant is selected from the group consisting of: Arabidopsis, maize, soybean, sunflower, sorghum, canola, mustard, wheat, alfalfa, cotton, rice, barley, millet, sugar cane and switchgrass.

16. The seed of claim 14, wherein said seed is selected from the group consisting of: Arabidopsis, maize, soybean, sunflower, sorghum, canola, mustard, wheat, alfalfa, cotton, rice, barley, millet, sugar cane and switchgrass.
